(19) United States
(12) Patent Application Publication
(10) Pub. No.: US 2022/0165379 A1
(43) Pub. Date: May 26, 2022

(54) METHODS AND SYSTEMS FOR REPORTING PATIENT- AND DRUG-SPECIFIC MEDICAL DATA

(71) Applicant: GENOMIND, INC., King of Prussia, PA (US)

(72) Inventors: Daniel DOWD, King of Prussia, PA (US); David J. ROBBINS, King of Prussia, PA (US); Stephen CLARKE, King of Prussia, PA (US); Ginette RANIERI, King of Prussia, PA (US)

(21) Appl. No.: 17/527,995

(22) Filed: Nov. 16, 2021

Related U.S. Application Data
(60) Provisional application No. 63/117,650, filed on Nov. 24, 2020.

(51) Int. Cl. G16H 15/00; G16H 10/60; G16H 20/70; G16H 20/10

(52) U.S. Cl. CPC G16H 15/00; G16H 20/10; G16H 20/70; G16H 10/60

(57) ABSTRACT

A method of generating a report presenting subject-specific information relevant to a treatment of a neuropsychiatric disorder includes obtaining a set of genetic test results. The set of genetic test results includes allelic information for each gene in a set of genomic loci. The method includes obtaining a set of medications including a first medication and obtaining a first patient-specific evaluation associated with the first medication using the set of genetic test results. The method includes determining a set of alternative medications to the first medication and determining, for each respective alternative medication of the set of alternative medications, a corresponding alternative pharmacokinetic and pharmacodynamic patient-specific evaluation using the set of genetic test results. The method further includes generating the report including the first medication, the first patient-specific evaluation, the set of alternative medications and, for each alternative medication in the set of alternative medications, the corresponding alternative patient-specific evaluation.

### CROSS-REFERENCE TO RELATED APPLICATIONS

This application claims priority to U.S. Provisional Patent Application No. 63/117,650, filed Nov. 24, 2020, entitled, “METHODS AND SYSTEMS FOR REPORTING PATIENT- AND DRUG-SPECIFIC MEDICAL DATA,” the content of which is hereby incorporated by reference, in its entirety, for all purposes.

### TECHNICAL FIELD

The present disclosure relates generally to methods and systems for reporting medical data.

### BACKGROUND

Not all patients react to a therapy in a uniform and beneficial manner. A number of factors including age, gender, ethnicity, and environmental and/or behavioral factors can influence the therapeutic efficacy and adverse reactions of therapeutic agents. Importantly, genetic variations among patients have been shown to account for variable drug reactions. Meyer, Urs A., “Pharmacogenetics and adverse drug reactions,” The Lancet 356:1667-71 (2000), Chad A. Bousman, “Pharmacogenetic tests and depressive symptom remission: a meta-analysis of randomized controlled trials,” NIH Pharmacogenomics 2019 January; 20(1):37-47. doi: 10.2217/pgs-2018-0142. For example, citalopram is one of the most commonly prescribed drugs for treating mental illness, such as depression. However, in populations with certain permutations of the gene CYP2C19 (2-4% Caucasians and 8-13% of Asians), administering a normal dosage of citalopram poses a significant risk of drug overexposure and adverse reaction. As such, a dosage adjustment may be necessary for these patients, as is reflected in the U.S. Food and Drug Administration's Prescribing Information (vide infra 0010). Thus, it is oftentimes beneficial for clinicians to have patients' pertinent genetic profiles available when making decisions, such as prescribing and dosing therapeutic agents.

Over the past 30 years, precision medicine has grown substantially, facilitated particularly by advances in molecular genetics and genotyping technologies. Modern genotyping technology allows for rapid detection and measurement of genetic variations, such as single nucleotide polymorphisms (SNPs), across a large span of the human genome. Over one-hundred million SNPs have been identified in the human population (Auton, A., et al., Nature, 526:68-74 (2015)), making them the most common type of genetic variation in humans. SNPs occur normally throughout the human genome and are mostly clinically insignificant. However, a relatively small portion of SNPs have been identified as important biomarkers associated with susceptibility to certain diseases and/or metabolism of different drugs. Syvanen, A., Nature Genetics, 37: S5-10 (2005). The SNP-based genotyping technology has been reportedly used in a variety of areas such as molecular diagnosis, prenatal analysis, predictive genetic testing, and in particular, pharmacotherapy, giving rise to the concept of “pharmacogenetics.” Roses, A., Nature, 405(3788):857-65 (2000).

Compared to conventional pharmaceutical approaches, where all patients diagnosed with a particular condition are prescribed a common therapy, pharmacogenetics provides personalized treatment based on the genotype profile specific to an individual patient. This allows for more accurate predictions about the patient's likely reaction to treatment. Accordingly, this approach helps clinicians achieve higher drug efficacy, increased drug tolerability, and reduced adverse reactions through a better selection of therapeutic agents with dosages optimized for the individual patient.

Because of the rapidly growing understanding of key genetic biomarkers, like SNPs, and the impact the biology underlying the biomarkers has on drug metabolism, many pharmaceuticals have FDA-approved labels that list pertinent genetic biomarkers, warnings particular to specific patient populations, and information about metabolism of the drug relative to such genetic biomarkers and warnings. FDA-approved labels commonly also contain information about pharmacokinetic and pharmacodynamic drug interactions. As can be found in the FDA, “Table of Pharmacogenomic Biomarkers in Drug labeling.” For example, the FDA-approved label for aripiprazole, an atypical antipsychotic drug used to treat schizophrenia and other mental disorders, states that “[d]osage adjustments are recommended in patients who are known CYP2D6 poor metabolizers and in patients taking concomitant CYP3A4 inhibitors or CYP2D6 inhibitors or strong CYP3A4 inducers.” ABILIFY® Prescribing information, Otsuka America Pharmaceutical Inc., 03US19IBR0002, (2019). These genes encode important enzymes that metabolize pharmaceuticals in the liver. As such, best medical practices warrant clinicians to consult with drug labels constantly for gene-drug association information and comply with the label's instructions.

For instance, mental illnesses are highly prevalent in the United States, and a major public health concern impacting nearly one in five adults. Serious mental illness (SMI), defined as a mental, behavioral, or emotional disorder resulting in serious functional impairment which substantially interferes with or limits one or more major life activities, is also prevalent in the U.S. More than 10 million adults, representing 4.2% of the adult U.S. population, have been diagnosed with SMI. NIH, Mental Illness, November 2017. SMI costs more than $193 billion per year in lost earnings in the U.S. Major depressive disorder (MDD), bipolar disorder, schizophrenia, schizoaffective disorder, and other SMIs are associated with increased mortality from various causes, including but not limited to suicide. John et al., Schizophr Res., 199:154-62 (2018); Laursen et al., J Clin Psychiatry, 68(6):899-907 (2007). In military veterans, post-traumatic stress disorder and traumatic brain injury also increase the risk of suicidal behavior. Wilks et al., J Psychiatr Res., 109:139-44 (2019). Because the distinction between serious and any mental illness is not always apparent, even mental illnesses that are not typically thought of as serious may be associated with excess mortality. For example, attention-deficit hyperactivity disorder (ADHD) is associated with excess mortality. In part, this may be due to co-morbidities, but this excess remains even when accounting for comorbid mental health diagnoses. The excess mortality in ADHD is driven mostly by unnatural causes, including accidents. Dalsgaard et al., Lancet, 385(9983):2190-96 (2015).

Many individuals suffering from SMI do not respond adequately or completely to initial therapy. For example, among patients with MDD, response to initial treatment fails to occur in approximately half of all individuals; remission is even less frequent. Trivedi M H et al., Am J Psych, 163:28-40 (2006). In MDD, work-related disability and productivity loss are critical determinants of patient quality of life and contribute significantly to the human and economic costs caused by this disease. Lee et al., J Affect Disord, 227:406-15 (2018). Schizophrenia, another SMI, follows a fairly consistent natural history characterized by an initial response to antipsychotic drugs, but subsequent non-adherence, deterioration and recurrent episodes of psychosis. Lieberman, J Clin Psychiatry, 67(10): e14 (2006).

In patients with SMI, pharmacogenetic testing has the potential to assist in the selection of drugs which are more likely well-tolerated, and to avoid serious adverse events (SAEs), as genetic variation is an important factor that influences the efficacy and tolerability (benefit: risk profile) of pharmaceutical agents, including psychotropic drugs. For example, cytochrome p450 (CYP450) enzymes account for the metabolism of most pharmaceuticals. The identification and validation of these pharmacokinetic genes and pharmacodynamic gene variants has enabled the emergence of precision medicine in psychiatry. Many pharmaceuticals, including many psychotropic drugs, now have biomarker warnings or precautions in their prescribing information, or contain pertinent information on the agent's metabolism, with respect to the effect of variants of genes encoding for CYP450 enzymes on the drug's exposure. Some product labels also contain information regarding the drug's ability to influence the exposure of concomitantly administered drugs via inhibition or induction of CYP450 enzymes. As the Agency notes, “Pharmacogenomics can play an important role in identifying responders and non-responders to medications, avoiding adverse events, and optimizing drug dose.” FDA, Table of Pharmacogenomic Biomarkers in Drug Labeling.

For example, aripiprazole, a second generation (or atypical) antipsychotic drug, is indicated to treat schizophrenia, mania associated with bipolar disorder, and several other serious disorders. Aripiprazole's label states: “Dosage adjustments are recommended in patients who are known CYP2D6 poor metabolizers and in patients taking concomitant CYP3A4 inhibitors or CYP2D6 inhibitors or strong CYP3A4 inducers.” Aripiprazole's label recommends half the usual starting dose in CYP2D6 poor metabolizers, and the dosage may vary by a factor of 8 in the presence of concomitant inducers or inhibitors of CYP450 enzymes. ABILIFY® Prescribing Information, 2018. Otsuka America Pharmaceutical Inc.

Inefficacy is an obvious potential consequence of underexposure. Alternatively, excessive exposure may be associated with common and manageable or infrequent and serious tolerability issues, such as orthostasis or tardive dyskinesia. One of the most common drugs used in psychiatry is citalopram. In CYP2C19 poor metabolizers (2-4% of Caucasians and 8-13% of Asians) the AUC exposure to citalopram may be doubled, increasing the risk of QT prolongation. CELEXA® (citalopram HBr) Prescribing Information. Forest Laboratories, Inc.

Other biomarkers may also have an important role in the safe use of psychotropics, including the avoidance of SAEs. One example is the presence of the HLA-B*1502 gene variant, which is associated with increased risk for severe and sometimes fatal skin reactions, such as Stevens-Johnson syndrome and toxic epidermal necrolysis, to carbamazepine and oxcarbazepine. Phillips et al., Clinical Pharmacogenetics Implementation Consortium Guideline for HLA Genotype and Use of Carbamazepine and Oxcarbazepine: 2017 Update, Clinical Pharmacology & Therapeutics (2018). Carbamazepine is indicated for epilepsy and trigeminal neuralgia but is widely utilized as a mood stabilizer in bipolar disorder. Carbamazepine extended-release capsules have an additional indication of acute mania or mixed episodes associated with bipolar I disorder. This risk is highlighted in carbamazepine's current drug label, which contains a boxed warning, and specifically calls for biomarker screening in individuals of Asian descent.

When treating patients with mental illnesses and disorders, it would be beneficial for clinicians to have a patient-specific report that can provide personalized treatment evaluations based on interpretive analysis of a patient's genotype. Although various research and clinical studies have looked for diagnostic and therapeutic indicators in an almost overwhelming variety of genomic markers, gene expression markers and protein markers, this vast and growing body of data has proven difficult to interpret. Synthesizing the tremendous amount of information on possible risk factors and indicators to apply this information clinically to diagnose and/or treat patients is challenging. As such, there is a need for methods and systems for providing patient-specific reports enabling medical professionals to apply the most relevant medical therapy in a meaningful manner to their patients.

### SUMMARY

Given the above background, what is needed in the art are methods and systems for providing medical professionals patient-specific reports for personalized treatment of neuropsychiatric disorders. The reports of the present disclosure provide for a method of reporting patient-specific information including pharmacogenetic test results obtained for the patient, patient-specific evaluations for the patient's current medications and/or planned medications, dosage guidelines, and alternative medicines to the patient's current or planned medications.

In accordance with some embodiments, a method of generating a report presenting subject-specific information relevant to a treatment of a neuropsychiatric disorder includes obtaining a set of genetic test results specific to a subject diagnosed with the neuropsychiatric disorder. The set of genetic test results includes allelic information for each gene in a set of genomic loci. The method includes obtaining a set of medications including a first medication for treatment of a first neuropsychiatric disorder in the plurality of neuropsychiatric disorders and obtaining a first patient specific evaluation associated with the first medication using all or a portion of the set of genetic test results. The method includes determining a set of alternative medications to the first medication for the treatment of the first neuropsychiatric disorder. The set of alternative medications and the first medication belong to a common therapeutic class. The method also includes determining, for each respective alternative medication of the set of alternative medications, a corresponding alternative patient-specific evaluation using at least all or a portion of the set of genetic test results. The method also includes generating the report. The report includes the first medication, the first patient-specific evaluation, the set of alternative medications, and for each alternative medication in the set of alternative medications, the corresponding alternative patient-specific evaluation.

In some embodiments, the first patient-specific evaluation is determined based on a risk of interaction between one or more genomic loci in the set of genomic loci and the first medication. The risk is identified by allelic information for one or more genomic loci in the set of genetic test results and an identity of the first medication.

In some embodiments, the first patient-specific evaluation is further determined based on one or more environmental modifiers associated with the subject.

In some embodiments, obtaining the first patient-specific evaluation includes retrieving information related to an interaction between the one or more genomic loci identified in the genetic test results and the first medication from a gene-drug interaction database.

In some embodiments, the set of medications includes a second medication distinct from the first medication. The first patient-specific evaluation associated with the first medication is further determined based on a combination of the first medication and the second medication. In some embodiments, the first medication and the second medication are from distinct therapeutic classes or from distinct drug classes.

In some embodiments, the respective patient-specific evaluation associated with the respective alternative medication is determined based on a risk of interaction between one or more genomic loci in the set of genomic loci and the respective alternative medication. The risk is identified by allelic information for one or more genomic loci in the set of genetic test results and an identity of the alternative medication.

In some embodiments, determining the corresponding patient-specific evaluation associated with the respective alternative medication includes retrieving information related to an interaction between the one or more genomic loci identified in the genetic test results, the respective alternative medication, and the one or more medications of the set of medications from a gene-drug interaction database.

In some embodiments, the set of medications includes a second medication distinct from the first medication. The respective patient-specific evaluation associated with the respective alternative medication is further determined based on a combination of the respective alternative medication and the second medication.

In some embodiments, creating the report includes ranking the first medication and each alternative medication in the set of alternative medications by corresponding patient-specific evaluation.

In some embodiments, determining the set of alternative medications is performed by selecting the one or more alternative medications, from among a plurality of medications that have an industry standard identifier associated with the first medication. In some embodiments, the set of alternative medications are from a different drug class than the first medication. In some embodiments, the industry standard identifier associated with the first medication includes information regarding ingredients, strength, and/or form of the first medication.

In some embodiments, the method further includes determining a first dosage modification recommendation for the first medication using all or a portion of the set of genetic test results.

In some embodiments, the method further includes determining a respective dosage modification recommendation for each respective alternative medications in the set of alternative medications using all or a portion of the set of genetic test results.

In some embodiments, the method further includes displaying, at a first portion of a graphical user interface, the genetic test results and displaying, at a second portion of the graphical user interface, a list including the set of medication.

In some embodiments, the method further includes displaying, in response to a user input on a first affordance, at a third portion of the graphical user interface, dosage information associated with the first medication.

In some embodiments, the method further includes displaying, in response to a user input on a second affordance, at the third portion of the graphical user interface, the report listing the first medication associated with the first patient-specific evaluation and the set of alternative medications. Each alternative medication in the set of alternative medications is associated with a respective patient-specific evaluation.

In some embodiments, the report includes one or more icons. Each icon of the one or more icons indicate a level of risk of the first patient-specific evaluation associated with the first medication and the respective patient-specific evaluation associated with the respective alternative medication.

In some embodiments, the method further includes receiving a user input selecting a first alternative medication from the set of alternative medications in the report and replacing the first alternative medication for the first medication

In some embodiments, the method further includes determining the set of genetic test results using a biological sample obtained from the subject. In some embodiments, the biological sample obtained from the subject includes buccal cells, saliva, or blood.

In some embodiments, the set of genomic loci is between one and twenty-four genomic loci. In some embodiments, the set of genomic loci comprises at least three genomic loci, at least four genomic loci, at least five genomic loci, or at least 10 genomic loci in Table 1 and/or Table 2.

In some embodiments, the set of genomic loci includes one or more genomic loci corresponding to a SNP selected from the group consisting of HTR2A rs7997012, 5HT2C rs3813929, ABCB1 C3435T rs1045642, ABCB1 rs2032583, ADRA2A rs1800544, ANK3 rs10994336, BDNF rs6265, CACNA1C rs1006737, COMT rs4680, rs2470890 (CYP1A2*1B), rs2069514 (CYP1A2*1C), rs35694136 (CYP1A2*1D), rs2069526 (CYP1A2*1E), rs762551 (CYP1A2*1F), rs12720461 (CYP1A2*1K), rs72547513 (CYP1A2*11), rs2279343 (CYP2B6*4), rs3211371 (CYP2B6*5), rs3745274 (CYP2B6*6), rs12248560 (CYP2C19*17), rs17884712 (CYP2C19*9), rs4244285 (CYP2C19*2), rs72552267 (CYP2C19*6), rs4986893 (CYP2C19*3), rs56337013 (CYP2C19*5), rs72558186 (CYP2C19*7), rs6413438 (CYP2C19*10), rs41291556 (CYP2C19*8), rs28399504 (CYP2C19*4), rs12769205 (CYP2C19*35), rs9332131 (CYP2C9*6), rs7900194 (CYP2C9*8 AND*27), rs1799853 (CYP2C9*2), rs1057910 (CYP2C9*3), rs28371686 (CYP2C9*5), rs56165452 (CYP2C9*4), rs28371685 (CYP2C9*11), rs72558187 (CYP2C9*13), rs35742686 (CYP2D6*3), rs5030656 (CYP2D6*9), rs1065852 (CYP2D6*10), rs16947 (CYP2D6*2), rs28371706 (CYP2D6*17), rs28371725 (CYP2D6*41), rs3892097 (CYP2D6*4), rs5030655 (CYP2D6*6), rs5030865 (CYP2D6*8 AND*14), rs59421388 (CYP2D6*29), rs774671100 (CYP2D6*15), rs5030862 (CYP2D6*12), rs5030863 (alternatively rs201377835, CYP2D6*11), rs5030867 (CYP2D6*7), CYP2D6 gene deletion, CYP2D6 gene multiplication, rs35599367 (CYP3A4*22), rs776746 (CYP3A5*3), rs10264272 (CYP3A5*6), rs41303343 (CYP3A5*7), DRD2 rs1799732, GRIK1 rs2832407, HLA-B*15:02 rs151107659, HLA-A*31:01, MC4R rs489693, MTHFR rs1801131 and rs1801133, OPRM1 rs1799971, SLC6A4 rs25531 and rs63749047, UGT1A4 rs2011425, and UGT2B15 rs1902023.

In some embodiments, the set of medications includes a SSRI or a tricyclic antidepressant (TCA). In some embodiments, the SSRI is citalopram, fluvoxamine, paroxetine, escitalopram or sertraline. In some embodiments, the TCA is amitriptyline.

In some embodiments, the set of medications comprises a serotonin-norepinephrine reuptake inhibitor (SNRI). In some embodiments, the SNRI is milnacipran or venlafaxine.

In some embodiments, the set of medications comprises a sodium channel modulating agent. In some embodiments, the sodium channel modulating agent is lamotrigine.

In some embodiments, the set of medications comprises an antipsychotic. In some embodiments, the antipsychotic is aripiprazole.

In some embodiments, the at least one neuropsychiatric disorder is depression, psychosis, or substance abuse. In some embodiments, the first neuropsychiatric disorder is substance abuse and the first medication is methadone. In some embodiments, the first neuropsychiatric disorder is depression and the first medication is mirtazapine.

Another aspect of the present disclosure provides for a non-transitory computer readable storage medium and one or more computer programs embedded therein. The one or more computer programs include instructions for generating a report presenting subject-specific information relevant to a treatment of a neuropsychiatric disorder. The instructions which, when executed by a computer system, cause the computer system to obtain a set of medications including a first medication for treatment of a first neuropsychiatric disorder in the plurality of neuropsychiatric disorders. The instructions cause the computer system to obtain a first patient-specific evaluation associated with the first medication using all or a portion of the set of genetic test results. The instructions cause the computer system to determine a set of alternative medications to the first medication for the treatment of the first neuropsychiatric disorder. The set of alternative medications and the first medication belong to a common therapeutic class. The instructions also cause the computer system to determine, for each respective alternative medication of the set of alternative medications, a corresponding alternative patient-specific evaluation using at least all or a portion of the set of genetic test results and to generate the report. The report includes the first medication, the first patient-specific evaluation, the set of alternative medications, and for each alternative medication in the set of alternative medications, the corresponding alternative patient-specific evaluation.

Yet another aspect of the present disclosure provides for a device for generating a report presenting subject-specific information relevant to a treatment of a neuropsychiatric disorder. The device includes one or more processors, and memory storing one or more programs for execution by the one or more processors. The one or more programs include instructions for obtaining a set of genetic test results specific to a subject diagnosed with the neuropsychiatric disorder. The set of genetic test results includes allelic information for each gene in a set of genomic loci. Each respective genomic loci in the set of genomic loci is associated with at least one neuropsychiatric disorder in a plurality of neuropsychiatric disorders. The one or more programs include instructions for obtaining a set of medications including a first medication for treatment of a first neuropsychiatric disorder in the plurality of neuropsychiatric disorders and obtaining a first patient-specific evaluation associated with the first medication using all or a portion of the set of genetic test results. The one or more programs include instructions for determining a set of alternative medications to the first medication for the treatment of the first neuropsychiatric disorder. The set of alternative medications and the first medication belong to a common therapeutic class. The one or more programs further include instructions for determining, for each respective alternative medication of the set of alternative medications, a corresponding alternative patient-specific evaluation using at least all or a portion of the set of genetic test results. The one or more programs also include instructions for also includes generating the report. The report includes the first medication, the first patient-specific evaluation, the set of alternative medications, and for each alternative medication in the set of alternative medications, the corresponding alternative patient-specific evaluation.

### BRIEF DESCRIPTION OF THE DRAWINGS

FIG. 1 illustrates a block diagram of an example computing device, in accordance with various embodiments of the present disclosure.

FIGS. 2A, 2B, 2C, 2D, and 2E are exemplary illustrations of a user interface for reporting patient-specific information relevant to a treatment of a neuropsychiatric disorder, in accordance with various embodiments of the present disclosure.

FIGS. 3A, 3B, 3C, 3D, and 3E illustrate flow charts describing a method for reporting patient-specific information relevant to a treatment of a neuropsychiatric disorder in accordance with various embodiments of the present disclosure.

Like reference numerals refer to corresponding parts throughout the several views of the drawings.

### DETAILED DESCRIPTION

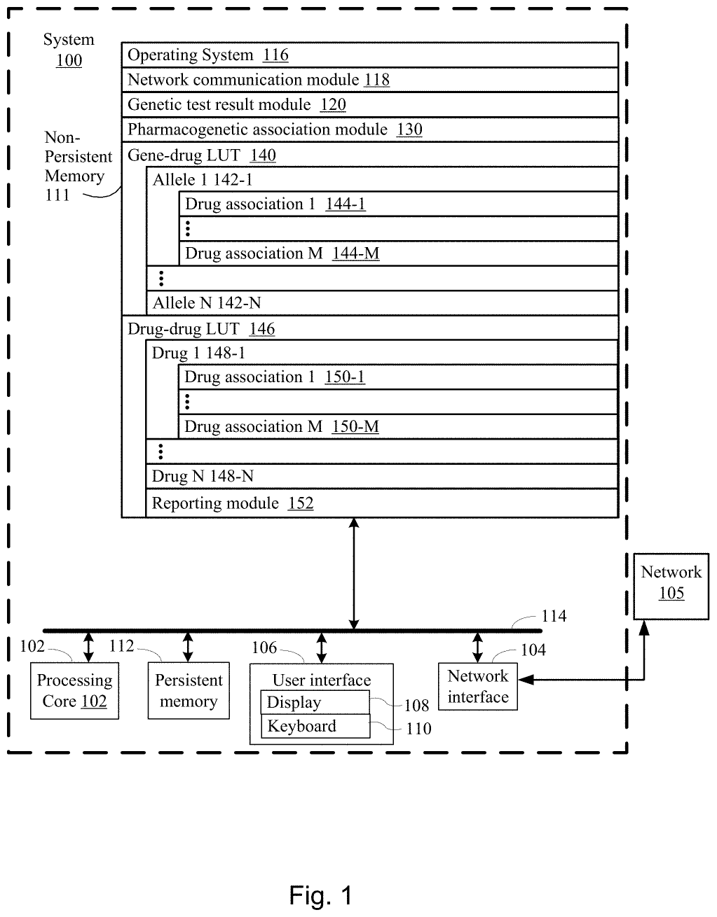

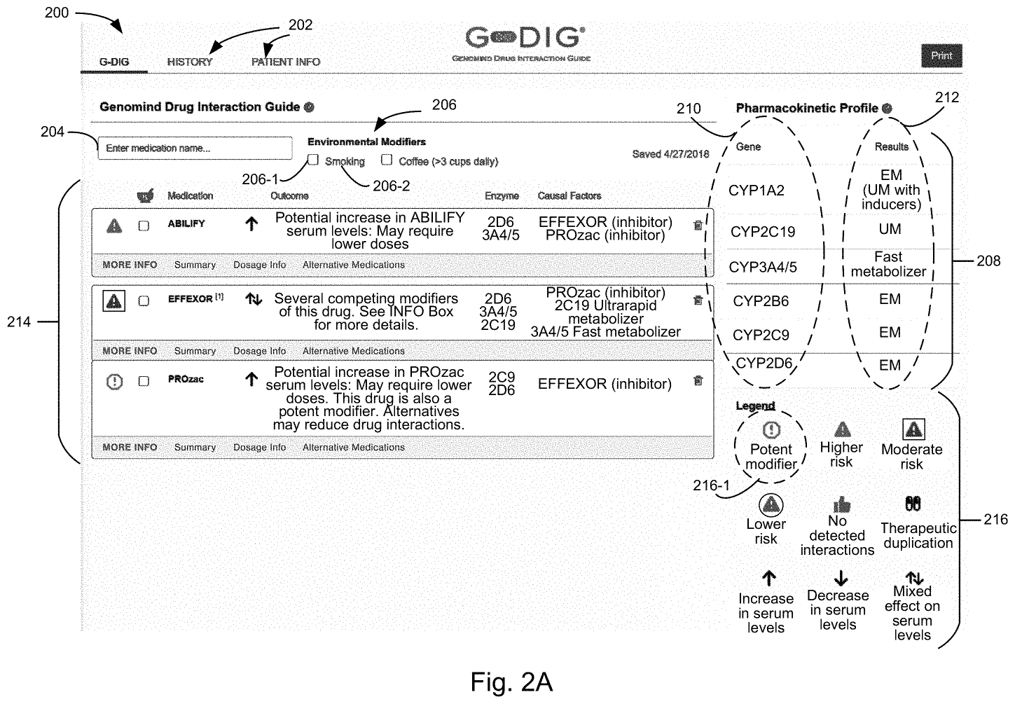

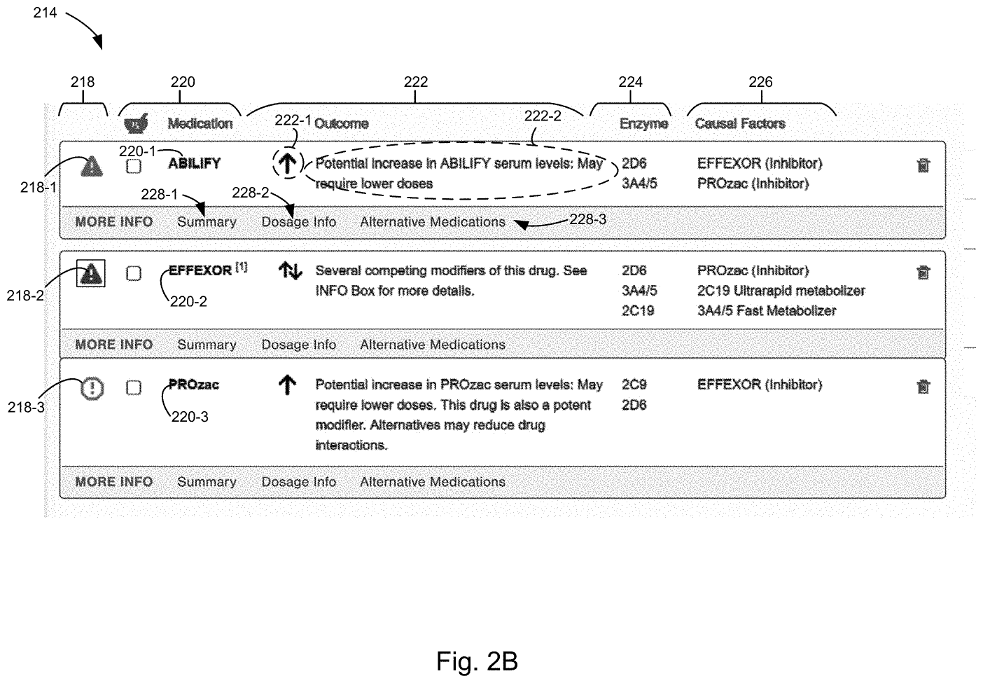

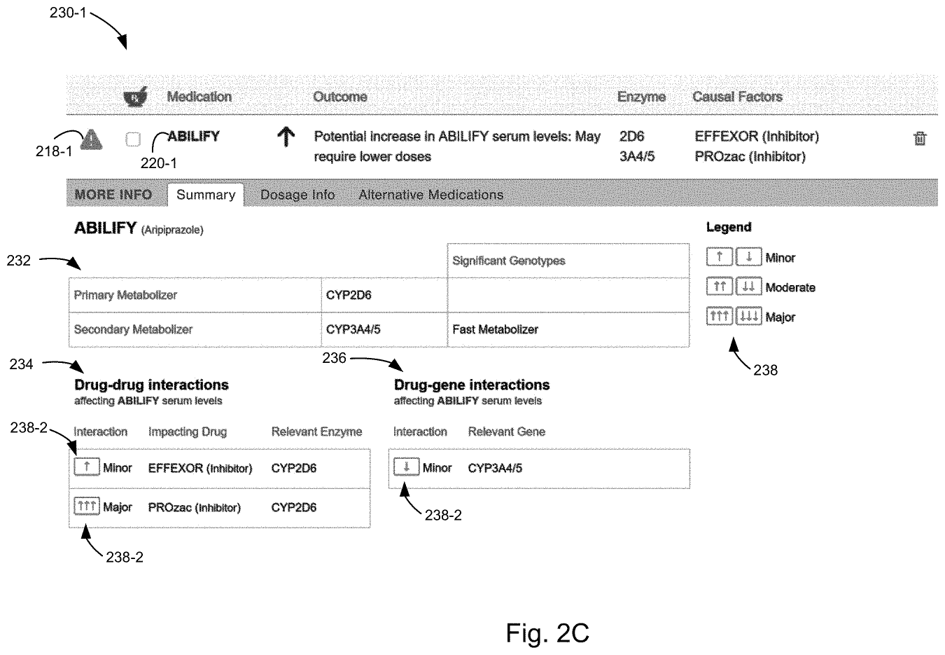

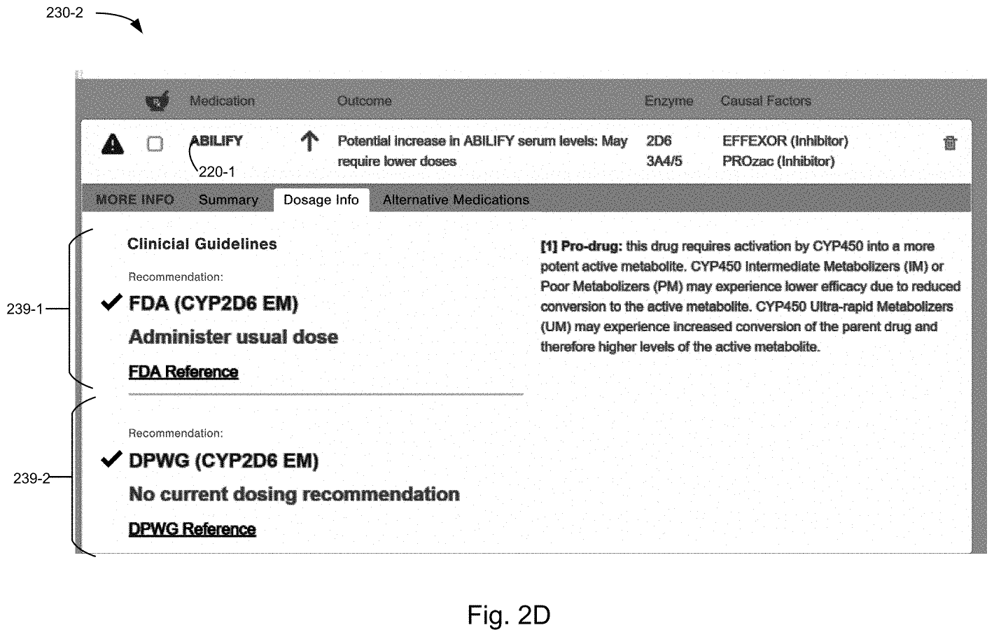

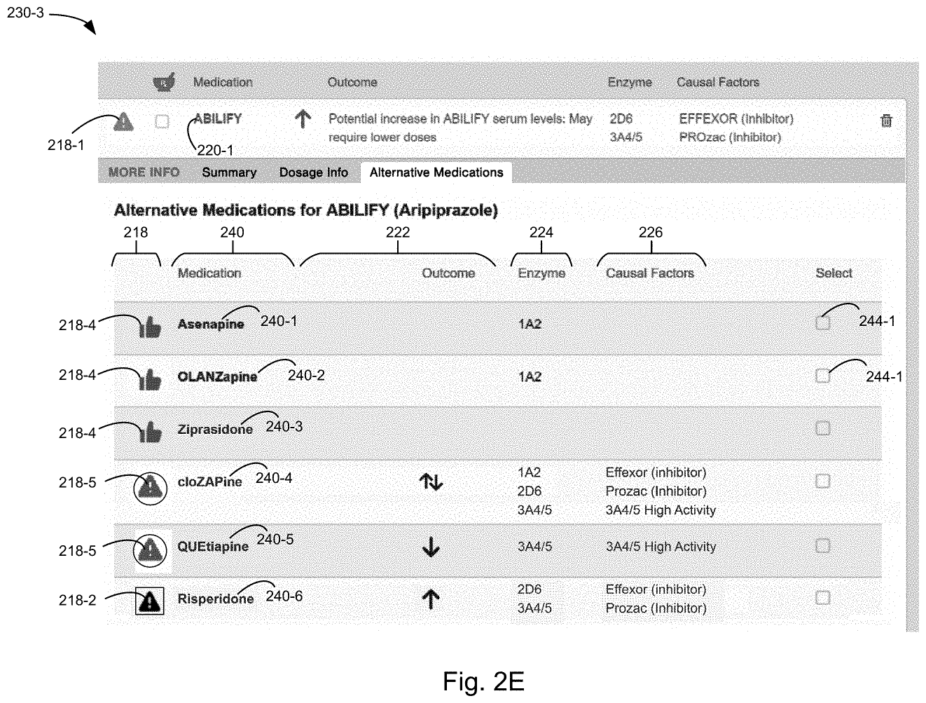

Referring to FIG. 1-FIG. 2E, the present disclosure provides for methods, systems and graphical user interfaces for reporting patient-specific information relevant to a treatment of a neuropsychiatric disorder.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first medication could be termed a second medication, and, similarly, a second medication could be termed a first medication, without departing from the scope of the present disclosure. The first medication and the second medication are both medications, but they are not the same medication. Furthermore, the terms “subject” and “patient,” “user” and “clinician,” and “drug” and “medication” are used interchangeably herein.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Example System Embodiments

Details of an example system are described in relation to FIG. 1. FIG. 1 is a block diagram illustrating system 100 for associating an allele status determined for a plurality of genomic loci with one or more evaluations for the treatment of a neuropsychiatric disorder and generating a patient-specific report comprising the one or more evaluations for the treatment of the neuropsychiatric disorder, in accordance with some implementations. System 100, in some implementations, includes one or more processing units CPU(s) 102 (also referred to as processors or processing cores), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid-state storage devices. The persistent memory 112 optionally includes one or more storage devices remotely located from the CPU(s) 102. The persistent memory 112, and the non-volatile memory device(s) within the non-persistent memory 111, comprise a non-transitory computer-readable storage medium. In some implementations, the non-persistent memory 111 or the non-transitory computer-readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 105;
a genetic test result module 120 for obtaining a set of genetic test result, including variant alleles (e.g., detected in a genotypic assay) measured from a patient and specific to one or more neuropsychiatric disorder;
a pharmacogenetic association module 130 for associating the variant alleles with evaluations for the management of the one or more neuropsychiatric disorder, e.g., using a gene-drug lookup table (LUT) 140 with drug associations 144 associated with each variant allele 142 and using a drug-drug LUT 146 with drug associations 150 associated with each drug 148; and
a reporting module 152 for generating a patient-specific report (e.g., in a form of a graphical user interface) comprising the one or more evaluations for the treatment of the neuropsychiatric disorder.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

Although FIG. 1 depicts a “system 100,” the figure is intended more as functional description of the various features, which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

Patient-Specific Reporting

As explained above, reviewing, evaluating, and interpreting a variety of diagnostic and therapeutic indicators including genomic markers, gene expression markers and protein markers together with information on risk factors and risk indicators creates a challenge for clinicians (e.g., users) treating patients with neuropsychiatric disorders. It is critical for clinicians to have access to a patient-specific report that can provide personalized treatment evaluations based on interpretive analysis of a patient's genotype.

The present disclosure provides for methods and systems for generating an interactive report including patient-specific information relevant to a treatment of a neuropsychiatric disorder. In some embodiments, the neuropsychiatric disorder is one of depression, psychosis, and substance abuse. The interactive report is provided to a clinician conveniently via a user interface (e.g., a graphical user interface). The report includes genetic test results obtained for the patient, the patient's current or planned medications, a patient specific evaluation for each of the patient's current or planned medications, and a set of alternative medications that can be considered to replace one or more of the patient's current or planned medications. In particular, the report provides a patient-specific evaluation and ranking for alternative medications. In some embodiments, the report further includes dosage evaluations associated with the patient's current medication and/or the set of alternative medications, consistent with published peer reviewed guidelines and/or FDA labels. Such patient-specific reports enable clinicians to review and evaluate the patient's medical therapy efficiently and conveniently for improved treatment of the neuropsychiatric disorder.

Creating the patient-specific report includes obtaining a set of genetic test results for the patient diagnosed with a neuropsychiatric disorder. In some embodiments, the set of genetic test results are determined from a biological sample obtained from the patient. The biological sample includes, for example, buccal cells, saliva, or blood. Exemplary methods and systems for obtaining the set of genetic test results from the biological sample are described in detail in U.S. Provisional Patent Application 62/969,906, entitled “Methods and Systems for Multiplex Allele Detection,” filed Feb. 4, 2020, the content of which is herein incorporated by its entirety. The set of genetic test results obtained are applied to guiding or modifying a course of therapy in an individual patient, including generating the patient-specific report for treating a neuropsychiatric disorder.

The set of genetic test results includes an allelic status of multiple genomic loci in a patient. In one embodiment, the set of genetic test results include screening across a plurality of genomic loci in one or more test subjects/patients for the presence of SNP markers listed in Table 1 and/or Table 2.

In some embodiments, a frequency of a most common allele for the first genomic locus in a population is at least 95% for a first genomic locus in the set of genomic loci. In some embodiments, a frequency of a second most common allele for the first genomic locus in the population is no more than 5% or no more than 10% for the first genomic locus in the set of genomic loci.

In some embodiments, the plurality of genomic loci associated with a neuropsychiatric disorder includes one or more genomic loci corresponding to a SNP selected from the group consisting of HTR2A rs7997012, 5HT2C rs3813929, ABCB1 C3435T rs1045642, ABCB1 rs2032583, ADRA2A rs1800544, ANK3 rs10994336, BDNF rs6265, CACNA1C rs1006737, COMT rs4680, rs2470890 (CYP1A2*1B), rs2069514 (CYP1A2*1C), rs35694136 (CYP1A2*1D), rs2069526 (CYP1A2*1E), rs762551 (CYP1A2*1F), rs12720461 (CYP1A2*1K), rs72547513 (CYP1A2*11), rs2279343 (CYP2B6*4), rs3211371 (CYP2B6*5), rs3745274 (CYP2B6*6), rs12248560 (CYP2C19*17), rs17884712 (CYP2C19*9), rs4244285 (CYP2C19*2), rs72552267 (CYP2C19*6), rs4986893 (CYP2C19*3), rs56337013 (CYP2C19*5), rs72558186 (CYP2C19*7), rs6413438 (CYP2C19*10), rs41291556 (CYP2C19*8), rs28399504 (CYP2C19*4), rs12769205 (CYP2C19*35), rs9332131 (CYP2C9*6), rs7900194 (CYP2C9*8 AND*27), rs1799853 (CYP2C9*2), rs1057910 (CYP2C9*3), rs28371686 (CYP2C9*5), rs56165452 (CYP2C9*4), rs28371685 (CYP2C9*11), rs72558187 (CYP2C9*13), rs35742686 (CYP2D6*3), rs5030656 (CYP2D6*9), rs1065852 (CYP2D6*10), rs16947 (CYP2D6*2), rs28371706 (CYP2D6*17), rs28371725 (CYP2D6*41), rs3892097 (CYP2D6*4), rs5030655 (CYP2D6*6), rs5030865 (CYP2D6*8 AND*14), rs59421388 (CYP2D6*29), rs774671100 (CYP2D6*15), rs5030862 (CYP2D6*12), rs5030863 (alternatively rs201377835, CYP2D6*11), rs5030867 (CYP2D6*7), CYP2D6 gene deletion, CYP2D6 gene multiplication, rs35599367 (CYP3A4*22), rs776746 (CYP3A5*3), rs10264272 (CYP3A5*6), rs41303343 (CYP3A5*7), DRD2 rs1799732, GRIK1 rs2832407, HLA-B*15:02 rs151107659, HLA-A*31:01, MC4R rs489693, MTHFR rs1801131 and rs1801133, OPRM1 rs1799971, SLC6A4 rs25531 and rs63749047, UGT1A4 rs2011425, and UGT2B15 rs1902023.

In some embodiments, the plurality of genomic loci includes any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70, of the genomic loci corresponding to a SNP selected from the group above. In some embodiments, the plurality of genomic loci includes at least three genomic loci, at least four genomic loci, at least five genomic loci, or at least 10 genomic loci in Table 1 and/or Table 2.

TABLE 1
Example SNPs with known pharmacogenetic associations
for the treatment of neuropsychiatric disorders.

| SNP | Position | Gene |
|---|---|---|
| rs7997012 | chr13: 46837850 | HTR2A |
| rs3813929 | chr10: 114584047 | HTR2C |
| rs1045642 | chr7: 87509329 | ABCB1 |
| rs2032583 | chr7: 87531245 | ABCB1 |
| rs1800544 | chr10: 111076745 | ADRA2A |
| rs6265 | chr11: 27658369 | BDNF |
| rs1006737 | chr12: 2236129 | CACNA1C |
| rs4680 | chr22: 19963748 | COMT |
| rs1799732 | chr11: 113475530 | DRD2 |
| rs2832407 | chr21: 29595188 | GRIK1 |
| rs489693 | chr18: 60215554 | MC4R |
| rs1801131 | chr1: 11794419 | MTHFR |
| rs1801133 | chr1: 11796321 | MTHFR |
| rs1799971 | chr6: 154039662 | OPRM1 |
| rs25531 | chr17: 30237328 | SLC6A4 |
| rs63749047 | chr17: 28564340 | SLC6A4 |

TABLE 2
Additional example SNPs with known pharmacogenetic associations
for the treatment of neuropsychiatric disorders.

| SNP | Position | Gene |
|---|---|---|
| rs2470890 | chr15: 74755085 | CYP1A2 |
| rs2069514 | chr15: 74745879 | CYP1A2 |
| rs35694136 | chr15: 74747272 | CYP1A2 |
| rs2069526 | chr15: 74749000 | CYP1A2 |
| rs762551 | chr15: 74749576 | CYP1A2 |
| rs72547513 | chr15: 74750296 | CYP1A2 |
| rs2279343 | chr19: 41009358 | CYP2B6 |
| rs3211371 | chr19: 41016810 | CYP2B6 |
| rs3745274 | chr19: 41006936 | CYP2B6 |
| rs4244285 | chr10: 94781859 | CYP2C19 |
| rs17878459 | chr10: 94775165 | CYP2C19 |
| rs4986893 | chr10: 94780653 | CYP2C19 |
| rs57081121 | chr10: 94780653 | CYP2C19 |
| rs28399504 | chr10: 94762706 | CYP2C19 |
| rs56337013 | chr10: 94852738 | CYP2C19 |
| rs72552267 | chr10: 94775453 | CYP2C19 |
| rs72558186 | chr10: 94781999 | CYP2C19 |
| rs41291556 | chr10: 94775416 | CYP2C19 |
| rs17884712 | chr10: 94775489 | CYP2C19 |
| rs6413438 | chr10: 94781858 | CYP2C19 |
| rs12248560 | chr10: 94761900 | CYP2C19 |
| rs12769205 | chr10: 94775367 | CYP2C19 |
| rs3758581 | chr10: 94842866 | CYP2C19 |
| rs1799853 | chr10: 94942290 | CYP2C9 |
| rs1057910 | chr10: 94981296 | CYP2C9 |
| rs56165452 | chr10: 94981297 | CYP2C9 |
| rs28371686 | chr10: 94981301 | CYP2C9 |
| rs9332131 | chr10: 94949282 | CYP2C9 |
| rs7900194 | chr10: 94942309 | CYP2C9 |
| rs28371685 | chr10: 94981224 | CYP2C9 |
| rs72558187 | chr10: 94941958 | CYP2C9 |
| rs1135840 | chr22: 42126611 | CYP2D6 |
| rs16947 | chr22: 42127941 | CYP2D6 |
| rs1135824 | chr22: 42129042 | CYP2D6 |
| rs35742686 | chr22: 42128242 | CYP2D6 |
| rs3892097 | chr22: 421289454 | CYP2D6 |
| rs5030655 | chr22: 42129084 | CYP2D6 |
| rs5030867 | chr22: 42127856 | CYP2D6 |
| rs5030865 | chr22: 42129033 | CYP2D6 |
| rs5030656 | chr22: 42128174 | CYP2D6 |
| rs1065852 | chr22: 42130692 | CYP2D6 |
| rs5030863 | chr22: 42525912 | CYP2D6 |
| rs5030862 | chr22: 42130668 | CYP2D6 |
| rs5030865(T) | chr22: 42129033 | CYP2D6 |
| rs774671100 | chr22: 42130655 | CYP2D6 |
| rs28371706 | chr22: 42129770 | CYP2D6 |
| rs61736512 | chr22: 42129132 | CYP2D6 |
| rs1058164 | chr22: 42129130 | CYP2D6 |
| rs59421388 | chr22: 42127608 | CYP2D6 |
| rs28371725 | chr22: 42127803 | CYP2D6 |
| rs35599367 | chr7: 99768693 | CYP3A4 |
| rs776746 | chr7: 99672916 | CYP3A5 |
| rs10264272 | chr7: 99665212 | CYP3A5 |
| rs41303343 | chr7: 99652771 | CYP3A5 |
| rs1061235 | chr6: 29945521 | HLA-A |
| rs151107659 | chr6: 32353777 | HLA-B |
| rs2011425 | chr2: 233718962 | UGT1A4 |
| rs1902023 | chr4: 68670366 | UGT2B15 |

User Interfaces for Interactive Reporting of Medical Information

FIGS. 2A-2D illustrate user interfaces (UI) (e.g., graphical user interfaces) for interactive reporting of patient-specific medical information. In particular, the user interfaces provide a comprehensive, convenient, and efficient manner for clinicians to review and evaluate the patient's pharmacogenetic profile, the patient's current or planned medications, as well as reviewing dosage recommendation and/or evaluating alternative medications. Importantly, the user interfaces provide risk evaluations and ranking for the alternative medications making the clinician's evaluating process more feasible. The user interfaces are interactive in that a clinician may, e.g., by providing user input on affordances (e.g., by clicking or providing a gesture on an icon or a tab), choose which information they wish to review. For example, the clinician may choose to review more detailed information regarding gene-drug interactions of the patient's current medications, review dosage guidelines for the patient's current medications, or compare the current medications against alternative medications.

FIGS. 2A-2E are exemplary illustrations of a user interface for reporting patient-specific information relevant to a treatment of a neuropsychiatric disorder, in accordance with various embodiments of the present disclosure. FIG. 2A presents an exemplary graphical user interface, UI 200, for interactive reporting of patient-specific medical information for a treatment of a neuropsychiatric condition. UI 200 includes a portion displaying a pharmacokinetic profile 208 of a patient. Pharmacokinetic profile 208 provides allelic information for a set of genes 210 listed in pharmacokinetic profile 208. The allelic information describes the patient's drug metabolism phenotypes with respect to set of genes 210. Each gene in set of genes 210 indicates a genomic locus corresponding to a SNP. For example, set of genes 210 in FIG. 2A include genomic loci corresponding to SNPs CYP1A2, CYP2C19, CYP3A4/5, CYP2B6, CYP2C9 and CYP2D6. Exemplary SNPs with known pharmacogenetic associations for the treatment of neuropsychiatric disorders are listed in Table 1 and Table 2 above. Pharmacokinetic profile 208 provides experimental test results 212 for each of the genomic loci identified in set of genes 210. Test results 212 correspond to identified metabolism phenotypes. The metabolism phenotypes are described in accordance with a categorization known in the art. For example, test results 212 identify whether the patient is categorized as a poor metabolizer (PM), intermediate metabolizer (IM), extensive metabolizer (EM), fast metabolizer (FM), or an ultra-rapid metabolizer (UM) with respect to each of the genomic loci identified in set of genes 210. Additional information may be provided, e.g., by identifying metabolizing phenotype with respect to inducers.

UI 200 also includes a portion for displaying information regarding a set medications (e.g., portion 214 including medications “Abilify,” “Effexor,” and “Prozac”). In some embodiments, the set of medications includes one or more medications of the patient's current medical therapy. In some embodiments, the set of medications includes one or more medications of the patient's planned medical therapy. Portion 214 displaying the first set of medications will be described in detail in FIG. 2B.

In some instances, UI 200 also includes an affordance 204 for adding medications (e.g., by typing a name or other identifier associated with a medication) to be included in portion 214. UI 200 further includes a section for identifying environmental modifiers 206 that may be relevant for the treatment of the patient. Such modifiers may include, for example, smoking, extensive caffeine consumption, exercise, or other information relevant to the patient's drug metabolism. A clinician may conveniently include environmental modifiers 206 to the report (e.g., to be taken into account in a patient-specific evaluation associated with medications) by clicking on an affordance (e.g., affordance 206-1) to include a respective modifier (e.g., modifier 206-2 titled “smoking”). UI 200 further includes a portion 216 including a set of icons (e.g., icon 216-1) with a respective explanation of a meaning of the icon. An icon is used as a visual indicator of a feature associated with a medication in portion 214. For example, icon 216-1 associated in “Prozac” in set of medications listed in portion 214 has a meaning of being a “potent modifier” as explained in section 216. A “potent modifier” refers to an identified gene-drug or drug-drug interaction that may alter the potency (e.g., drug activity expressed in terms of the amount of drug required to produce an effect of given intensity) of the respective medication.

In some embodiments, UI 200 further includes alternative displays, e.g., alternative tabs or pop-up windows (e.g., tabs 202), that allows a clinician to efficiently and conveniently access additional information relevant for the treatment of the patient. In some instances, the clinician may click on a “History” tab that reports information regarding the patient's medical therapy and/or other treatments or therapies (e.g., previous test results, previous medications, previous non-medical therapies, etc.) or by clicking on “Patient Info” tab that reports additional information regarding the patient (e.g., biometric data and/or any other information relevant for the treatment of the patient).

FIG. 2B illustrates portion 214 of UI 200 including a set of medications 220 and information associated with set of medications 220 (e.g., icons 218 indicating possible risks associated with respective medications, information about drug-gene interactions outcome 222, identification of relevant enzymes 224, and information regarding possible causal factors 226). In some embodiments, set of medications 220 includes one or more medications that the patient is currently being treated for. In some embodiments, set of medications 220 includes one or more medications of a planned medical treatment for the patient. In some embodiments, set of medications 220 includes medications relevant to a treatment of one or more neuropsychiatric conditions that the patient is diagnosed with (e.g., depression, psychosis, or substance abuse). In some embodiments, set of medications 220 includes medications relevant for treatment of other conditions that the patient has been diagnosed with. Set of medications 220 is obtained by a user input. For example, a clinician inputs set of medications 220 using affordance 204 in FIG. 2A. As another example, the clinician imports or downloads set of medication 220 from a database.

In some embodiments, the neuropsychiatric condition is depression. In some embodiments, set of medication 220 includes an SSR1 or a tricyclic antidepressant (TCA). In some embodiments, the SSR1 is citalopram, fluvoxamine, paroxetine, escitalopram or sertraline. In some embodiments, the TCA is amitryptyline. In some embodiments, set of medications 220 includes seratonin-norepinephrine reuptake inhibitor (SNR1). In some embodiments, the SNR1 is milnacipran or venlafzine. In some embodiments, set of medications 220 includes sodium channel modulating agent. In some embodiments, the sodium channel modulating agent is lamotrigine.

In some embodiments, the neuropsychiatric condition is psychosis and set of medications 220 includes an antipsychotic medicine. In some embodiments, set of medications 220 includes clozapine.

In some embodiments, the neuropsychiatric disorder is substance abuse and set of medications 220 includes methadone or bupropion. In some embodiments, the neuropsychiatric disorder is depression and first medication is mertazapine.

In FIG. 2B, set of medications 220 includes first medication 220-1 (“Abilify”), second medication 220-2 (“Effexor”), and third medication 220-3 (“Prozac”). Each medication in set of medications 220 is associated with a patient-specific evaluation. A patient-specific evaluation indicates a level of risk associated with a respective medication based on at least gene-drug interaction and/or drug-drug interactions. The risk assessments provide information on whether the identifier gene-drug or drug-drug interactions may influence the patient's drug metabolism and therefore the serum levels associated with the respective medication. The patient-specific evaluation is determined based on at least pharmacokinetic profile 208 of the patient and the respective medication in set of medications 220. In particular, the patient-specific evaluation is determined based on at least interactions between the respective medication and one or more genomic loci identified in pharmacokinetic profile 208. The gene-drug interactions are determined and reported based on curated literature. In some embodiments, the gene-drug information is retrieved from Food and Drug Administration's (FDA), Dutch Pharmacokinetics Working Group (DPWG), and/or Clinical Pharmacogenetics Implementation Consortium (CPIC®). In some embodiments, system 100 described with respect to FIG. 1 retrieves curated literature information of gene-drug interactions from FDA, DPWG, and/or CPIC®. The gene-drug interaction information is saved and organized into a database (e.g., gene-drug lookup table 140) under non-persistent memory 111. Determining and reporting a patient-specific evaluation associated with a particular medication in the set of medications 220 includes retrieving relevant information from gene-drug lookup table 140 based on the respective medication and the patient's pharmacokinetic profile 208.

In some embodiments, the patient-specific evaluation also includes drug-drug interactions. The drug-drug interactions are determined and reported based on curated literature. In some embodiments, the drug-drug information is retrieved from FDA, DPWG, and/or CPIC®) databases. The drug-drug interaction information is saved and organized into a database (e.g., drug-drug lookup table 146) under non-persistent memory 111. Determining and reporting a patient-specific evaluation associated with a particular medication in the set of medications 220 includes retrieving relevant information from drug-drug lookup table 146 based on the respective medication and other medications in set of medication 220. For example, a patient-specific evaluation associated with first medication 220-1 includes patient-specific evaluation based on an interaction of first medication 220-1 with medication 220-2 and/or medication 220-3.

In some embodiments, the patient-specific evaluation is further determined based on one or more environmental modifiers (e.g., one or more environmental modifiers 206 identified by the clinician FIG. 2A). The information related to interactions between one or more environmental modifiers 206 and set of medications 220 and/or pharmacokinetic profile is based on the curated literature. As described before, determining and reporting a patient-specific evaluation including environmental modifiers includes retrieving relevant information from gene-drug lookup table 140 based on the respective medication, the patient's pharmacokinetic profile 208, and the environmental modifiers 206.

For example, a first patient-specific evaluation for first medication 220-1 is determined by identifying, from gene-drug lookup table 140, all gene-drug interactions that first medication 220-1 may have with the genomic loci and associated phenotype included in the patient's pharmacokinetic profile 208. Any information with respect to the identified gene-drug interactions are retrieved from gene-drug lookup table 140. For example, the information includes a description of the gene-drug interaction and/or guidance related to the gene-drug interaction. The information associated with first medication 220-1 is then reported by displaying information relevant to the patient-specific evaluation in portion 214 of UI 200.

The patient-specific evaluation for respective medications in set of medications 220 includes reporting outcome 222 including description 222-2 providing a description and/or guidance with respect to the respective patient-specific evaluation. For example, the description and/or guidance uses language retrieved from the curated literature. Outcome 222 also includes an outcome icon 222-1 providing a visual indicator of an effect that the drug has on relevant serum levels. For example, outcome icon 222-1 may indicate that there is an increase in the serum levels (e.g., an upward arrow), a decrease in the serum levels (e.g., a downward arrow), or a mixed effect on the serum levels (e.g., a combination of an upward arrow and a downward arrow). In some instances, patient-specific evaluation also includes reporting the relevant enzymes (e.g., enzymes 224) and identified causal factors (e.g., causal factors 226). An overall level of the risk is indicated with an icon (e.g., risk level icons 218). For example, first medicine 220-1 is associated with a risk level indicated with icon 218-1 (“potent inhibitor”), second medicine 220-2 is associated with a risk level indicated with icon 218-2 (“moderate risk”), and third medicine 220-3 is associated with a risk level indicated with icon 218-3 (‘high risk”). For example, a respective icon 218 has a color, a shape, and/or a symbol indicating a level of risk associated with the respective medication. Explanations associated with icons 218 are displayed in portion 216 of UI 200 described above with respect to FIG. 2A.

UI 200 further provides a clinician a convenient manner for reviewing additional information associated with each of the medications in set of medications 220. As shown in FIG. 2A, portion 214 of UI 200 displays affordances (e.g., affordances 228) associated with each of the medications in set of medication 220. For example, affordances 228-1, 228-2, and 228-3 are associated with first medication 220-1. By receiving a user input (e.g., a click with a mouse) on any of these affordances, UI 200 displays additional sections (e.g., additional tabs or pop-up windows) that provide additional information and interactive features useful for a treatment of the patient. In some instances, affordance 228-1 is associated with summary tab 230-1 described with respect to FIG. 2C below, affordance 228-2 is associated with dosage information tab 230-2 described with respect to FIG. 2D below, and affordance 228-3 is associated with alternative medications tab 230-3 described with respect to FIG. 2E below.

FIG. 2C illustrates summary tab 230-1 of UI 200 that displays information related to first medication 220-1 of set of medications 220. In some embodiments, summary tab 230-1 provides additional information about first medication 220-1 including information about the metabolizing enzymes, (e.g., section 232), information about drug-drug interactions (e.g., section 234), and information about drug-gene interactions (e.g., section 236). As shown, section 232 displays a table including metabolizer enzymes, including a primary metabolizer (e.g., CYP2D6) and a secondary metabolizer (e.g., CYP3A4/5), associated with first medication 220-1 (“Abilify”). In some instances, section 232 also displays any significant genotypes associated with these metabolizers. For example, the secondary metabolizer CYP3A4/5 enzyme is identified as a “Fast Metabolizer” in section 232. In some instances, section 234 displays a table including information about drug-drug interactions that may affect serum levels of first medication 220-1. The drug-drug interactions include interactions of first medication 220-1 with other medications in set of medications 220, including second medication 220-2 (“Effexor”) and third medication 220-3 (“Prozac”). For example, the table in section 234 identifies relevant enzymes for each of the interactions, a function of each of the interactions. In FIG. 2C, the table of section 234 indicates that second medication 220-2 and third medication 220-3 are inhibitors if the serum levels of first medication 220-1. The strength of the interaction is further classified as minor, moderate, or major. The strength of the interaction is indicated with icons 238-1 and 238-2 for each of the medications. Explanation for the icons 238-1 and 238-2 is provided in section 238 of summary tab 230-1. Tab 230-1 further includes section 236 including a table identifying relevant drug-gene interactions affecting the serum levels of first medication 220-1. For example, the table in section 236 includes relevant genes (e.g., CYP3A4/5) from set of genes 210 identified in the patient's pharmacokinetic profile 208 in FIG. 2A. The table of section 236 icon 238-3 for indicated the strength of the respective drug-gene interaction.

Table 2D illustrates dosage information tab 230-2 displaying dosage information and/or guidance relevant to of first medication 220-1. The dosage information and/or guidance for first medication 220-1 is reported based on curated literature (e.g., from FDA, CPIC®, and/or DPWG databases). The information is included, for example, in gene-drug lookup table 140 and/or drug-drug lookup table 146 of system 100 in FIG. 1. In some embodiments, the information is included in and retrieved from an additional lookup table, e.g., a dosage lookup table, in non-persistent memory 111 of system 100. For example, in FIG. 2D, information from an FDA database is displayed in section 239-1 including a recommendation (e.g., “administer usual dose”) together with a description (e.g., “[1]Pro-drug: this drug requires activation by CYP450 into a more potent active metabolite . . . ). Information retrieved from a DPWG database is displayed in section 239-2. However, in this instance DPWG database included no dosing recommendation for first medication 220-1 (e.g., “no current dosing recommendation”).

As described above with respect to FIG. 2C, first medication 220-1 (“Abilify”) is associated with a high risk level determined based on at least the drug-drug interactions 243 with second medication 220-2 (“Effexor”) and third medication 220-3 (“Prozac”) and on drug-gene interactions between first drug 220-1 and the patient's pharmacokinetic profile 208 in FIG. 2A. The high risk level is indicated, for example, with icon 218-1 indicating that first medication is a “Potent Inhibitor.” Because of the determined high risk level, a clinician may want to review and evaluate one or more alternative medications to first medication 220-1 in order to determine whether any alternative medication would provide a lower risk level while providing a same therapeutic effect. In some embodiments, the one or more alternative medications are from a same therapeutic class. A therapeutic class refers to a set of medications that are categorized to have a common therapeutic use. Medications belonging to a same therapeutic class may have different chemistry but are used to treat a same pathology. For example, medications belonging to a particular therapeutic class are intended for treatment of a common pathology but are chemically distinct. In some embodiments, the therapeutic class is antidepressant or antipsychotic. In some embodiments, the one or more alternative medications are from a distinct drug class. A drug class refers to a set of medications and/or other therapeutic compounds that have similar chemical structures and the same therapeutic mechanism and/or closely related therapeutic mechanism. In some instances, medications of a particular drug class are used for treating a common disease.

Curated literature databases, including, e.g., FDA, DPWG, and CPIC databases, include alternative medication schemes for medications and/or therapeutic compounds. However, reviewing and evaluating such databases, with respect to the current medications the patient is treated with (e.g., medications 220 in FIG. 2B), the patient's pharmacokinetic profile (e.g., pharmacokinetic profile 208 in FIG. 2A) and/or environmental modifiers (e.g., environmental modifiers 206) is challenging due to the complexity of the data. The present disclosure provides for a convenient, thorough, and efficient manner for a clinician to review alternative medications to the current one or more medications that the patient is being treated with.

FIG. 2E illustrates alternative medications tab 230-3 that allows the clinician to review and evaluate a plurality of alternative medications against a current or planned medication in set of medications 220 (e.g., medication 220-1). Alternative medications tab 230-3 displays a table including one or more alternative medications 240. As described above, alternative medications 240 associated with first medication 220-1 are determined based on at least curated literature databases. In some embodiments, alternative medications 240 are determined by identifying, from curated literature databases, any medication having a common industry standard identifier as first medication 220-1 has. An industry standard identifier refers to an identifier belonging to a normalized naming system for generic and branded medications. The normalized naming system provides normalized names (e.g., identifiers) for all approved clinical drugs and links the names to medicine vocabularies and databases known in the art. In some embodiments, the industry standard identifier includes information regarding ingredients, strength, and/or form of the associated medicine. In some embodiments, the industry standard identifier is a US Pharmacopeia Drug Classification System entry. In some embodiments, the industry standard identifier refers to RxNorm naming system managed by The National Library of Medicine (NML). For example, the industry standard identifier associated with first medication 220-1 corresponds to an RxNorm name of first medication 220-1 and the RxNorm name of first medication includes information regarding ingredients, strength and/or form of first medication 220-1. In some embodiments, alternative medications 240 are associated with a same industry standard identifier as first medication 220-1. The alternative medications 240 have a same therapeutic class but are from a different drug class. Alternative medications 220 therefore treat the same neuropsychiatric condition as first medication 220-1 but have a different chemical structure and may therefore have a different mechanism for treatment of the neuropsychiatric condition. In some embodiments, alternative medications 220 therefore may have different drug-gene interactions. Furthermore, because of the different chemical structure and different mechanism, alternative medications may have different drug-drug interaction with other medications in set of medications 220 (e.g., second medication 220-2 and third medication 220-3). One or more of alternative medications 240 may therefore provide an improved or comparable therapeutic effect as first medication 220-1 with a lower patient-specific evaluation.

In FIG. 2E, alternative medications 240 include alternative medications including alternative medication 240-1 (“Asenapine”), alternative medication 240-2 (“Olanzapine”), alternative medication 240-3 (“Ziprasidone”), alternative medication 240-4 (“Clozapine”), alternative medication 240-5 (“Quetiapine”), and alternative medication 240-6 (“Risperidone”). Each alternative medication of alternative medications 240 is associated with a respective alternative medication patient-specific evaluation. Similar to a patient-specific evaluation associated with respective medications 220, in some instances alternative patient-specific evaluation for respective alternative medications 240 includes reporting outcome 222 providing a description and/or guidance with respect to the respective patient-specific evaluation. For example, the description and/or guidance includes language retrieved from the curated literature. Outcome 222 may also include outcome icon 222-1 providing a visual indicator of an effect that the drug has on relevant serum levels (e.g., an upward arrow, a downward arrow and/or a combination of an upward arrow and a downward arrow). The alternative medicine patient-specific evaluation also includes reporting the relevant enzymes (e.g., enzymes 224) and identified causal factors (e.g., causal factors 226) associated with respective alternative medications 240. An overall level of risk is indicated with an icon (e.g., risk level icons 218). For example, alternative medications 240-1, 240-2, and 240-3 are associated with a risk level indicated with icon 218-4 (“no detected interactions”) while alternative medications 240-4 and 240-5 are associated with a risk level indicated with icon 218-1 (“lower risk”) and alternative medication 240-6 is associated with a risk level indicated with icon 218-2 (moderate risk). Explanations for the icons associated with 218 are displayed in portion 216 of UI 200 described above with respect to FIG. 2A.

In some embodiments, alternative medications 240 displayed in alternative medications tab 330-3 are ranked in accordance with the level of risk each alternative medication is associated with. In some embodiments, each of the alternative medications 240 is categorized based on the respective patient-specific evaluation. The categories include first category titled “no detectable interaction,” a second category titled “lower risk,” a third category titled “moderate risk,” a fourth category titled “higher risk,” and a fifth category titled “potent modifier.” Alternative medications 240 are then listed in the table displayed in tab 230-3 in accordance with the ranking so that the first category is displayed first and the fifth category is displayed last. For example, in FIG. 2A alternative medications 240-1, 240-2, and 240-3 are associated with the first category titled “no detectable interactions.” The alternative medicines in the first category are considered to correspond to the lowest risk alternative medications and are therefore displayed first (highest) in the table of alternative medications tab 330-1. Consequently, alternative medications 240-4 and 240-5 associated with the second category “lower risk” are displayed after the alternative medication associated with the first category. Alternative medication 240-6 is associated with the third category titled “moderate risk,” and is therefore displayed after the first category (e.g., alternative medication 240-5 is displayed lowest in alternative medications tab 230-3).

In some embodiments, set of medications 220 includes one or more compound medications (e.g., including one or more therapeutic ingredients). In such embodiments, alternative medications are determined and reported for each of the therapeutic ingredients in the compound medication. The alternative medications may include compound medication and/or medication with just one therapeutic ingredient.

As shown in FIG. 2E, alternative medications tab 230-3 provides for a convenient and efficient way for a clinician to review and evaluate alternative medications 240 against first medication 220-1. As shown in FIG. 2E, the clinician may easily observe that all of the alternative medications 240 are associated with a lower risk than first medicine 220-1 which is ranked in the fifth category “potent modifier.” For example, alternative medication 240-1 (“Asenapine”), alternative medication 240-2 (“Olanzapine”), alternative medication 240-3 (“Ziprasidone”) are all ranked under the first category “no detectable interactions,” and may thereby be more preferable medications for the treatment of the patient.

Alternative medications tab 230-3 further allows a clinician to replace first medication 220-1 with an alternative medication by providing a user input on an affordance associated with each of alternative medication 240. For example, the clinician may click on affordance 244-1 associated with alternative medication 240-1 “Asenapine.” First medication 220-1 is then replaced with alternative medication 240-1 and UI 200 described with respect to FIGS. 2A-2E is updated to display information, guidance, dosage, and alternative medications associated with alternative medication 240-1 instead of first medication 220-1. The clinician may further select any of the alternative medications to be replaced with alternative medication 240-1 and the clinician may repeat reviewing and selecting alternative medications for second medication 220-2 and/or alternative medication 220-3 as well. UI 200, therefore, provides for an easy and fast method of reviewing and evaluating a complex set of information that is relevant for a treatment of the patient's neuropsychiatric condition.

Methods for Reporting Medical Data

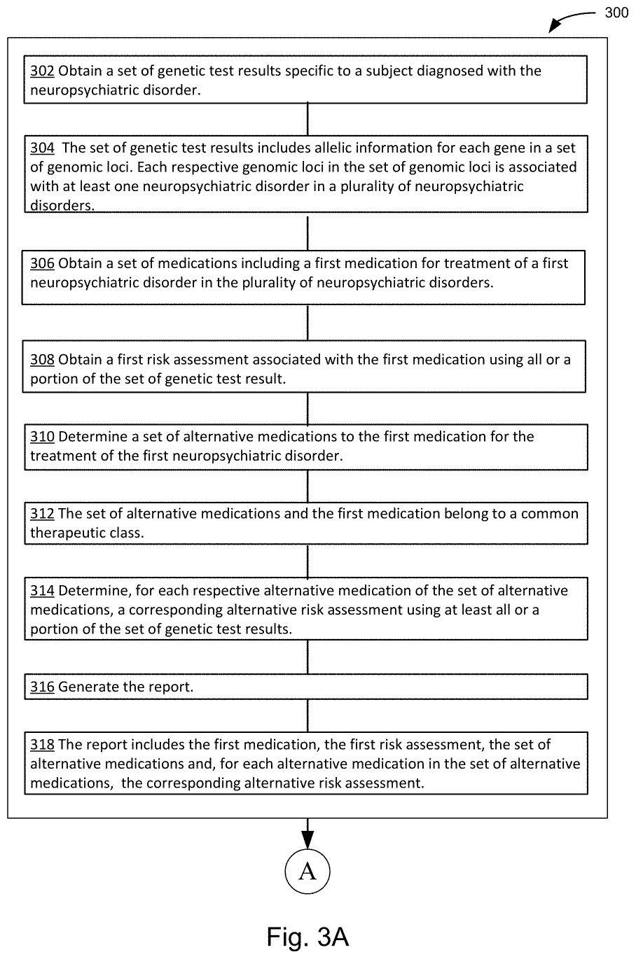

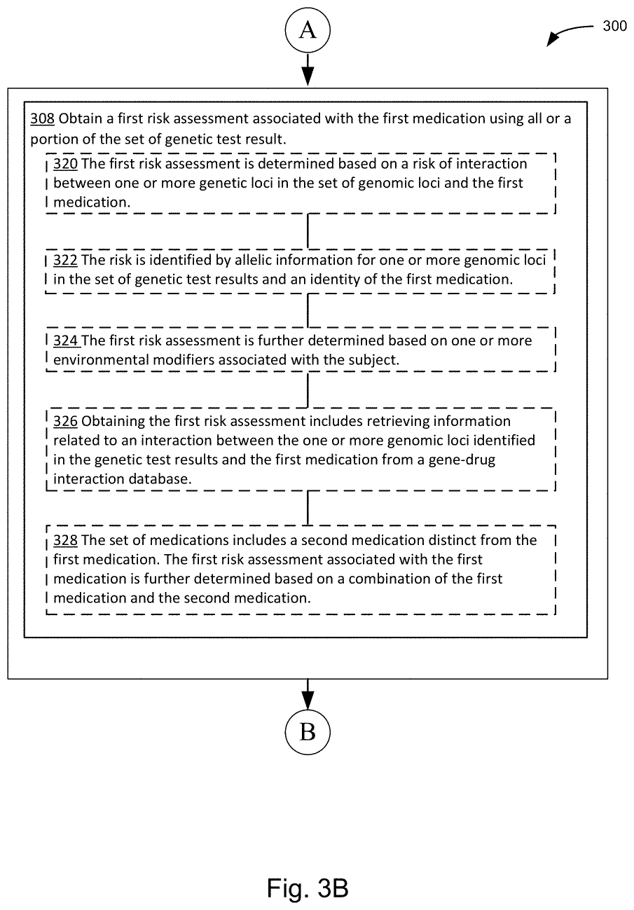

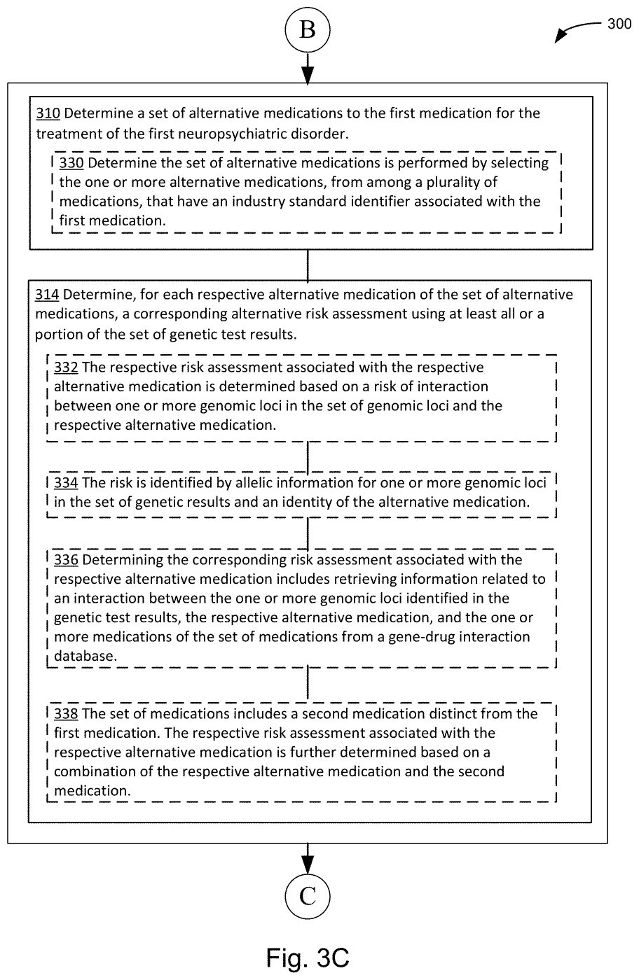

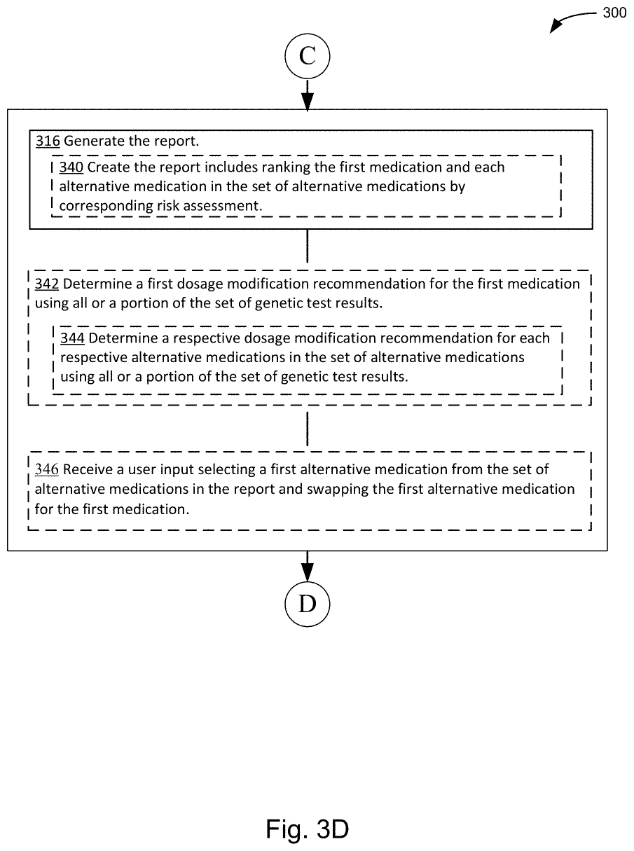

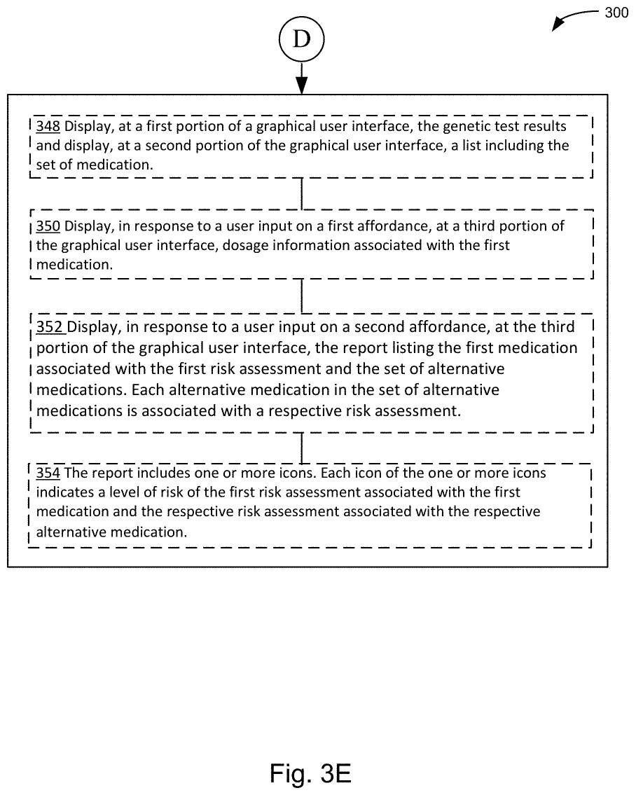

Now that details of UI 200 for reporting patient-specific information relevant to a treatment of a neuropsychiatric disorder are disclosed, details regarding methods and features of reporting the patient-specific information, in accordance with an embodiment of the present disclosure, are disclosed. FIGS. 3A-3E illustrate flow charts describing method 300 of generating a report presenting subject-specific (e.g., patient-specific) information relevant to a treatment of a neuropsychiatric disorder, in accordance with some embodiments. Optional blocks are indicated with dashed boxes in FIGS. 3A-3E. Method 300 provides for an interactive, efficient, and convenient way for a user (e.g., a clinician) to review and evaluate the subject's pharmacokinetic profile, risks associated with the subject's current medication, dosage evaluations and guidelines, and alternative medications for the subject's current or planned medications. In some embodiments, such methods and features are carried out by reporting module 152 of system 100 described above with respect to FIG. 1.

Block 302. With reference to block 302 of FIG. 3A, method 300 includes obtaining a set of genetic test results specific to a subject (e.g., a patient) diagnosed with the neuropsychiatric disorder. In some embodiments, the neuropsychiatric disorder is one of depression, psychosis, and substance abuse. In some embodiments, the set of genetic test results corresponds to a pharmacokinetic profile of the subject. For example, UI 200 displays pharmacokinetic profile 208 obtained for a subject in FIG. 2A. Pharmacokinetic profile 208 reports experimentally obtained test results 212 for set of genes 210. A pharmacokinetic profile provides information related to a patient-specific chemical metabolism of specific drugs (e.g., drugs included in the set of medications).

In some embodiments, method 300 includes determining the set of genetic test results using a biological sample obtained from the subject. The biological sample includes buccal cells, saliva or blood. In some embodiments, obtaining the set of genetic test results includes any of the methods and systems described in U.S. Provisional Patent Application 62/969,906, entitled “Methods and Systems for Multiplex Allele Detection,” filed Feb. 4, 2020, the content of which is herein incorporated by its entirety.

Block 304. With reference to block 304 of FIG. 3A, the set of genetic test results includes allelic information for each gene in a set of genomic loci. For example, pharmacokinetic profile 208 includes phenotype categorization (e.g., test results 212) of drug metabolism for a set of genomic loci (e.g., genomic loci identified as set of genes 210). Each respective genomic loci in the set of genomic loci is associated with at least one neuropsychiatric disorder in a plurality of neuropsychiatric disorders. In some embodiments, the set of genetic test results include 1-24 genomic loci. For example, pharmacokinetic profile 208 including the allelic information for set of genes 210 is associated with a neuropsychiatric disorder that the patient is diagnosed with.

Block 306. With reference to block 306 of FIG. 3A, method 300 includes obtaining a set of medications including a first medication for the treatment of a first neuropsychiatric disorder in the plurality of neuropsychiatric disorders. In some embodiments, the set of medications includes the subject's current medications. In some embodiments, the set of medications includes medications that are part of a planned medical treatment for the subject. In some embodiments, the set of medications is obtained by an input from the user. For example, a user enters set of medications FIG. 2A by a user input on affordance 204 (e.g., by entering the medications name) or by importing set of medications from a database.

Block 308. With reference to block 308 of FIG. 3A, method 300 includes obtaining a first patient-specific evaluation associated with the first medication using all or a portion of the set of genetic test results. Obtaining the first patient-specific evaluation is described with respect to FIG. 3B below.

Block 310. With reference to block 310 of FIG. 3A, method 300 includes determining a set of alternative medications to the first medication for the treatment of the first neuropsychiatric disorder. The set of alternative medications includes one or more alternative medications. The set of alternative medications and the first medication belong to a common therapeutic class (Block 312). The alternative medications to the first medication are thereby applied for treatment of the neuropsychiatric disorder (e.g., depression, psychosis, or substance abuse) that the patient is diagnosed with. In some embodiments, the set of alternative medications includes alternative medications that are from a distinct drug class than the first medication. The alternative medications therefore have a same therapeutic effect as the first medication but have different chemical structures than the first medication. In some instances, the set of alternative medications includes alternative medications that are from the same drug class than the first medication but are distinct from the first medication such that the alternative medicine patient-specific evaluations associated with such alternative medications are distinct from the first patient-specific evaluation.

In some embodiments, the neuropsychiatric condition is depression. In some embodiments, the set of medications and/or the set of alternative medications includes a SSR1 or a tricyclic antidepressant (TCA). In some embodiments, the SSR1 is citalopram, fluvoxamine, paroxetine, escitalopram or sertraline. In some embodiments, the TCA is amitryptyline. In some embodiments, the set of medications and/or the set of alternative medications includes seratonin-norepinephrine reuptake inhibitor (SNR1). In some embodiments, the SNR1 is milnacipran or venlafzine. In some embodiments, the set of medications and/or the set of alternative medications includes a sodium channel modulating agent. In some embodiments, the sodium channel modulating agent is lamotrigine.

In some embodiments, the neuropsychiatric condition is psychosis and the set of medications and/or the set of alternative medications includes an antipsychotic medicine. In some embodiments, the set of medications and/or the set of alternative medications includes clozapine.

In some embodiments, the neuropsychiatric disorder is substance abuse and the set of medications and/or the set of alternative medications includes methadone or bupropion. In some embodiments, the neuropsychiatric disorder is depression and first medication is mertazapine.

Block 314. With reference to block 314 of FIG. 3A, method 300 includes determining, for each respective alternative medication of the set of alternative medications, a corresponding alternative patient-specific evaluation using at least all or a portion of the set of genetic test results. Determining the corresponding alternative patient-specific evaluation is described with respect to FIG. 3C below.

Block 316. With reference to block 316 of FIG. 3A, method 300 includes generating the report. The report identifies at least the first set of medications including the first medication, the first patient-specific evaluation, the set of alternative medications, and for each alternative medication in the set of alternative medications the corresponding alternative patient-specific evaluation (Block 318). Features of the report are described with respect to UI 200 of FIGS. 2A-2E in accordance with some embodiments. For example, portion 214 of UI 200 displays set of medications 220 including first medication 220-1 in FIG. 2B. By providing a user input on affordance 228-3, a user may review set of alternative medications 240 for first medication 220-1, as is described with respect to FIG. 2E.

Block 320. With reference to block 320 of FIG. 3B, the first patient-specific evaluation is determined based on a risk of interaction between one or more genomic loci in the set of genomic loci (e.g., set of genes 210 in pharmacokinetic profile 208 in FIG. 2A) and the first medication (e.g., first medication 220-1 in FIG. 2B). The risk is identified by allelic information for one or more genomic loci in the set of genetic test results (e.g., test results 212 identify drug metabolism phenotypes associated with set of genes 210) and an identity of the first medication (Block 322).

In some embodiments, the set of genomic loci is between one and twenty-four genomic loci. In some embodiments, the set of genomic loci comprises at least three genomic loci, at least four genomic loci, at least five genomic loci, or at least 10 genomic loci in Table 1 and/or Table 2. In some embodiments, for a first genomic locus in the set of genomic loci, a frequency of a most common allele for the first genomic locus in a population is at least 95%. In some embodiments, for a first genomic locus in the set of genomic loci, a frequency of a second most common allele for the first genomic locus in the population is no more than 10%, or no more than 5%.

In some embodiments, the set of genomic loci includes one or more genomic loci corresponding to a SNP selected from the group consisting of HTR2A rs7997012, 5HT2C rs3813929, ABCB1 C3435T rs1045642, ABCB1 rs2032583, ADRA2A rs1800544, ANK3 rs10994336, BDNF rs6265, CACNA1C rs1006737, COMT rs4680, rs2470890 (CYP1A2*1B), rs2069514 (CYP1A2*1C), rs35694136 (CYP1A2*1D), rs2069526 (CYP1A2*1E), rs762551 (CYP1A2*1F), rs12720461 (CYP1A2*1K), rs72547513 (CYP1A2*11), rs2279343 (CYP2B6*4), rs3211371 (CYP2B6*5), rs3745274 (CYP2B6*6), rs12248560 (CYP2C19*17), rs17884712 (CYP2C19*9), rs4244285 (CYP2C19*2), rs72552267 (CYP2C19*6), rs4986893 (CYP2C19*3), rs56337013 (CYP2C19*5), rs72558186 (CYP2C19*7), rs6413438 (CYP2C19*10), rs41291556 (CYP2C19*8), rs28399504 (CYP2C19*4), rs12769205 (CYP2C19*35), rs9332131 (CYP2C9*6), rs7900194 (CYP2C9*8 AND*27), rs1799853 (CYP2C9*2), rs1057910 (CYP2C9*3), rs28371686 (CYP2C9*5), rs56165452 (CYP2C9*4), rs28371685 (CYP2C9*11), rs72558187 (CYP2C9*13), rs35742686 (CYP2D6*3), rs5030656 (CYP2D6*9), rs1065852 (CYP2D6*10), rs16947 (CYP2D6*2), rs28371706 (CYP2D6*17), rs28371725 (CYP2D6*41), rs3892097 (CYP2D6*4), rs5030655 (CYP2D6*6), rs5030865 (CYP2D6*8 AND*14), rs59421388 (CYP2D6*29), rs774671100 (CYP2D6*15), rs5030862 (CYP2D6*12), rs5030863 (alternatively rs201377835, CYP2D6*11), rs5030867 (CYP2D6*7), CYP2D6 gene deletion, CYP2D6 gene multiplication, rs35599367 (CYP3A4*22), rs776746 (CYP3A5*3), rs10264272 (CYP3A5*6), rs41303343 (CYP3A5*7), DRD2 rs1799732, GRIK1 rs2832407, HLA-B*15:02 rs151107659, HLA-A*31:01, MC4R rs489693, MTHFR rs1801131 and rs1801133, OPRM1 rs1799971, SLC6A4 rs25531 and rs63749047, UGT1A4 rs2011425, and UGT2B15 rs1902023.

In some embodiments, the first patient-specific evaluation is further determined based on one or more environmental modifiers associated with the subject (Block 324). In some embodiments, the environmental modifiers include daily caffeine consumption (e.g., an indication that the subject's daily caffeine consumption is above a threshold level for caffeine consumption), smoking, alcohol, other medical substances, and/or other factors influencing drug metabolism.

Block 326. With reference to block 330 of FIG. 3C, obtaining the first patient-specific evaluation includes retrieving information related to an interaction between the one or more genomic loci identified in the genetic test results and the first medication from a gene-drug interaction database (Block 326). For example, the gene-drug interaction database corresponds to gene-drug lookup table 140 stored in non-persistent memory 111 of system 100 in FIG. 1. Gene-drug lookup table 140 includes information collected from curated literature databases. In some embodiments, the gene-drug interaction database includes information retrieved from FDA, CPIC® and/or DPWG gene-drug interaction databases. In some embodiments, the set of medications includes a second medication (e.g., second medication 220-2 in FIG. 2B) distinct from the first medication (Block 328). The first patient-specific evaluation associated with the first medication is further determined based on a combination of the first medication and the second medication. For example, the first patient-specific evaluation associated with first medication 220-1 also takes into account drug-drug interactions between first medication 220-1 and second medication 220-2 and/or third medication 220-3. Such a feature is significant for evaluating the total effect of the subject's current or planned medical therapy to the metabolism of first medication 220-1. In some embodiments, determination of the first patient-specific evaluation based on the combination of the first medication and the second medication includes retrieving information related to a drug-drug interaction between the first medication and the second medication from a drug-drug interaction database. For example, the drug-drug interaction database corresponds to drug-drug lookup table 146 stored in non-persistent memory 111 of system 100 in FIG. 1. Drug-drug lookup table 146 includes information collected from curated literature databases, such as FDA, CPIC® and/or DPWG drug-drug interaction databases.

Block 330. With reference to block 330 of FIG. 3C, determining the set of alternative medications is performed by selecting the one or more alternative medications from among a plurality of medications that have an industry standard identifier associated with the first medication. An industry standard identifier refers to an identifier belonging to a normalized naming system for generic and branded medications. The normalized naming system provides normalized names (e.g., identifiers) for all approved clinical drugs and links the names to medicine vocabularies and databases known in the art (e.g., FDA, CPIC and/or DPWG databases). In some embodiments, the industry standard identifier includes information regarding ingredients, strength, and/or form of the associated medicine. In some embodiments, the industry standard identifier refers to RxNorm naming system managed by The National Library of Medicine (NML). In some embodiments, the industry standard identifier associated with the first medication includes information regarding ingredients, strength, and/or form of the first medication.

Block 332. With reference to block 332 of FIG. 3C, the respective patient-specific evaluation associated with the respective alternative medication is determined based on a risk of interaction between one or more genomic loci in the set of genomic loci and the respective alternative medication. The risk is identified by allelic information for one or more genomic loci in the set of genetic results and an identity of the alternative medication (Block 334). In some embodiments, determining the corresponding patient-specific evaluation associated with the respective alternative medication includes retrieving information related to an interaction between the one or more genomic loci identified in the genetic test results, the respective alternative medication, and the one or more medications of the set of medications from a gene-drug interaction database (Block 336). In some embodiments, the information is retrieved from gene-drug lookup table 140 and/or drug-drug lookup table 146.

In some embodiments, the set of alternative medications (e.g., alternative medications 240) is from a different drug class than the first medication. The alternative medications, therefore, have different chemical structure than the first medication. The chemical structure of a medication is a significant factor in the metabolism of the medication. Therefore, a subject may metabolize one medicine without significant gene-drug interactions while has a gene-drug interaction interfering metabolism of another medicine.

In some embodiments, the set of medications includes a second medication distinct from the first medication (e.g., set of medications 220 includes first medication 220-1 and second medication 220-2) (Block 338). The respective patient-specific evaluation associated with the respective alternative medication is further determined based on a combination of the respective alternative medication and the second medication. In some embodiments, the first medication and the second medication are from distinct therapeutic classes. In some embodiments, the first medication and the second medication are from distinct drug classes. In some embodiments, the first and the second medication are from the same therapeutic class but have different chemical structures. In some embodiments, the second medication and the respective alternative medication are from different therapeutic classes or from different drug classes.

Block 340. With reference to block 340 of FIG. 3D, creating the report includes ranking the first medication and each alternative medication in the set of alternative medications by corresponding patient-specific evaluation. For example, in FIG. 2E, each of first medication 220-1 and alternative medications 240-1, 240-2, 240-3, 240-4, 240-5, and 240-6 are associated with a respective patient-specific evaluation. A patient-specific evaluation is assigned to a risk category in accordance significance of the impact indicated by the patient-specific evaluation. The categories include, for example, “no detected interactions,” “lower risk,” “moderate risk,” “higher risk,” and “potent modifier.” The categories are ranked in an order of the impact on the subject's serum levels such that “no detected interactions” indicates the lowest risk possible (e.g., the gene-drug interactions and/or the drug-drug interaction have no detected impact on serum levels of the associated medicine). “Lower risk” risk indicates detected interactions but with a low impact on the serum levels, “moderate risk” indicates detected interactions with a moderate impact on the serum levels, and “higher risk” indicates detected interactions with a high level of impact on the serum levels. “Potent modifier” is ranked as the highest risk. Icons 218 for each of the medications indicates a level of the risk determined based on the patient-specific evaluation (see, explanations of different icons in portion 216 in FIG. 2A). In some embodiments, the alternative medications are ranked in accordance with the risk level they are associated with. For example, in FIG. 2E first medication 220-1 is associated with risk level “potent modifier” indicated with icon 218-1, alternative medications 240-1, 240-2, and 240-3 are associated with risk level “no detected interaction” indicated with icons 218-4, alternative medications 240-4 and 240-5 are associated with risk level “lower risk” indicated with icons 218-5, and 240-6 is associated with risk level “moderate risk” indicated with icon 218-2. Alternative medications 240 are ranked in an order based on the risk levels such that alternative medications 240-1, 240-2, and 240-3 with the lowest patient-specific evaluation “no detected interactions” are displayed first and alternative medications 240-4 and 240-5 with the patient-specific evaluation “lower risk” are displayed second, and alternative medication 240-6 with “moderate risk” is displayed third.

Block 342. With reference to block 342 of FIG. 3D, method 300 includes determining a first dosage modification recommendation for the first medication using all or a portion of the set of genetic test results. For example, tab 230-2 in FIG. 2D includes dosage evaluations in sections 239-1 and 239-2 for first medication 220-1. In some embodiments, the first dosage recommendation is determined based on curated literature information, such as FDA (e.g., FDA dosage recommendation in section 239-1), CPIC and/or DPWG (e.g., DPWG dosage recommendation in section 239-2) databases. Method 300 also includes determining a respective dosage modification recommendation for each respective alternative medications in the set of alternative medications using all or a portion of the set of genetic test results (Block 344).

Block 346. With reference to block 346 of FIG. 3D, method 300 includes receiving a user input selecting a first alternative medication from the set of alternative medications in the report and replacing the first alternative medication for the first medication. For example, a user can select alternative medication 240-1 by providing a user input on affordance 244-1 in FIG. 2E. In accordance with this selection, first medication 220-1 is replaced by alternative medication 240-1 and UI 200 is updated to reflect this replacement throughout FIGS. 2A-2E. Similarly, a user may select to replace second medication 220-2 and/or third medication 220-3 as well. In this way, the user can easily evaluate multiple therapeutic plans for the subject in a simple and fast manner.

Block 348. With reference to block 348 of FIG. 3E, method 300 includes displaying, at a first portion of a graphical user interface, the genetic test results (e.g., pharmacokinetic profile 208 of UI 200 in FIG. 2A) and display, at a second portion of the graphical user interface, a list including the set of medications (e.g., portion 214 of UI 200 displays a table including set of medications 220 in FIG. 2B). Method 300 includes displaying, in response to a user input on a first affordance (e.g., a tab or an icon), at a third portion of the graphical user interface, dosage information associated with the first medication (Block 350). In some embodiments, displaying the third portion of the graphical user interface includes displaying a tab or a pop-up window including the dosage information associated with the first medication. For example, a user provides an input (e.g., by clicking with a mouse or providing a touch gesture) on affordance 228-2 in FIG. 2B thereby opening a tab or a pop-up window (e.g., dosage information tab 230-2) displaying dosage information and guidance for first medication 220-1. Method 300 includes displaying, in response to a user input on a second affordance, at the third portion of the graphical user interface, the report listing the first medication associated with the first patient-specific evaluation and the set of alternative medications (Block 352). For example, a user provides an input (e.g., by clicking with a mouse) on affordance 228-3 in FIG. 2B thereby opening a tab or a pop-up window (e.g., alternative medication tab 230-3) displaying alternative medications 230 for first medication 220-1. Each alternative medication in the set of alternative medications is associated with a respective patient-specific evaluation. The report includes one or more icons (Block 354). Each icon of the one or more icons (e.g., icons 218 in FIG. 2E) indicates a level of risk of the first patient-specific evaluation associated with the first medication and the respective patient-specific evaluation associated with the respective alternative medication. For example, the icon has a color, shape, and/or symbol indicating a level of risk associated with the respective medication.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces described with respect to FIGS. 2A-2E. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of generating a report presenting subject-specific information relevant to a treatment of a neuropsychiatric disorder or associated comorbidity, the method comprising:
obtaining a set of genetic test results specific to a subject diagnosed with the neuropsychiatric disorder or associated comorbidity, wherein the set of genetic test results comprises allelic information for each gene in a set of genomic loci, wherein each respective genomic locus in the set of genomic loci is associated with at least one treatment option for a neuropsychiatric disorder in a plurality of neuropsychiatric disorders;
obtaining a set of medications including a first medication for treatment of a first neuropsychiatric disorder in the plurality of neuropsychiatric disorders;
obtaining a first patient-specific evaluation associated with the first medication using all or a portion of the set of genetic test results;
determining a set of alternative medications to the first medication for the treatment of the first neuropsychiatric disorder, wherein the set of alternative medications and the first medication belong to a common therapeutic class;
determining, for each respective alternative medication of the set of alternative medications, a corresponding alternative patient-specific evaluation using at least all or a portion of the set of genetic test results; and
generating the report, wherein the report comprises the first medication, the first patient-specific evaluation, the set of alternative medications and, for each alternative medication in the set of alternative medications, the corresponding alternative patient-specific evaluation.

2. The method of claim 1, wherein:
the first patient-specific evaluation is determined based on a risk of interaction between one or more genomic loci in the set of genomic loci and the first medication, wherein the risk is identified by allelic information for one or more genomic loci in the set of genetic test results and an identity of the first medication, and wherein
obtaining the first patient-specific evaluation includes retrieving information related to an interaction between the one or more genomic loci identified in the set of genetic test results and the first medication from a gene-drug interaction database.

3. The method of claim 2, wherein the first patient-specific evaluation is further determined based on one or more environmental modifiers associated with the subject.

4. The method of claim 2, wherein the set of medications includes a second medication distinct from the first medication, and the first patient-specific evaluation associated with the first medication is further determined based on a combination of the first medication and the second medication, wherein the first medication and the second medication are from distinct therapeutic classes or from distinct drug classes.

5. The method of claim 4, wherein the respective patient-specific evaluation associated with the respective alternative medication is determined based on a risk of interaction between one or more genomic loci in the set of genomic loci and the respective alternative medication, wherein the risk is identified by allelic information for one or more genomic loci in the set of genetic results and an identity of the alternative medication, and wherein
determining the corresponding patient-specific evaluation associated with the respective alternative medication includes retrieving information related to an interaction between the one or more genomic loci identified in the set of genetic test results, the respective alternative medication, and the one or more medications of the set of medications from a gene-drug interaction database.

6. The method of claim 5, wherein the set of medications includes a second medication distinct from the first medication, and the respective patient-specific evaluation associated with the respective alternative medication is further determined based on a combination of the respective alternative medication, and the second medication.

7. The method of claim 1, wherein creating the report includes ranking the first medication and each alternative medication in the set of alternative medications by corresponding patient-specific evaluation.

8. The method of claim 1, wherein determining the set of alternative medications is performed by selecting one or more alternative medications, from among a plurality of medications, that have an industry standard identifier associated with the first medication, wherein the set of alternative medications are from a different drug class than the first medication, and the industry standard identifier associated with the first medication includes information regarding ingredients, strength, and/or form of the first medication.

9. The method of claim 1, further including:
determining a first dosage modification recommendation for the first medication using all or a portion of the set of genetic test results, and
determining a respective dosage modification recommendation for each respective alternative medications in the set of alternative medications using all or a portion of the set of genetic test results.

10. The method of claim 1, further including:
displaying, at a first portion of a graphical user interface, the genetic test results;
displaying, at a second portion of the graphical user interface, a list including the set of medication;
displaying, in response to a user input on a first affordance, at a third portion of the graphical user interface, dosage information associated with the first medication; and
displaying, in response to a user input on a second affordance, at the third portion of the graphical user interface, the report listing the first medication associated with the first patient-specific evaluation and the set of alternative medications, each alternative medication in the set of alternative medications associated with a respective patient-specific evaluation, wherein the report includes one or more icons, each icon of the one or more icons indicating a level of risk of the first patient-specific evaluation associated with the first medication and the respective patient-specific evaluation associated with the respective alternative medication.

11. The method of claim 1, further including receiving a user input selecting a first alternative medication from the set of alternative medications in the report and replacing the first alternative medication for the first medication.

12. The method of claim 1, wherein the set of genomic loci is between one and twenty-five genomic loci and comprises at least three genomic loci, at least four genomic loci, at least five genomic loci, or at least 10 genomic loci in Table 1 and/or Table 2.

13. The method of claim 1, further comprising determining the set of genetic test results using a biological sample obtained from the subject comprising buccal cells, saliva, or blood.

14. The method of claim 1, wherein the set of genomic loci comprises human alleles corresponding to the SNPs rs5030863, rs28371685, and rs5030867.

15. The method of claim 1, wherein the set of genomic loci comprises one or more genomic loci corresponding to a SNP selected from the group consisting of HTR2A rs7997012, 5HT2C rs3813929, ABCB1 C3435T rs1045642, ABCB1 rs2032583, ADRA2A rs1800544, ANK3 rs10994336, BDNF rs6265, CACNA1C rs1006737, COMT rs4680, rs2470890 (CYP1A2*1B), rs2069514 (CYP1A2*1C), rs35694136 (CYP1A2*1D), rs2069526 (CYP1A2*1E), rs762551 (CYP1A2*1F), rs12720461 (CYP1A2*1K), rs72547513 (CYP1A2*11), rs2279343 (CYP2B6*4), rs3211371 (CYP2B6*5), rs3745274 (CYP2B6*6), rs12248560 (CYP2C19*17), rs17884712 (CYP2C19*9), rs4244285 (CYP2C19*2), rs72552267 (CYP2C19*6), rs4986893 (CYP2C19*3), rs56337013 (CYP2C19*5), rs72558186 (CYP2C19*7), rs6413438 (CYP2C19*10), rs41291556 (CYP2C19*8), rs28399504 (CYP2C19*4), rs12769205 (CYP2C19*35), rs9332131 (CYP2C9*6), rs7900194 (CYP2C9*8 AND*27), rs1799853 (CYP2C9*2), rs1057910 (CYP2C9*3), rs28371686 (CYP2C9*5), rs56165452 (CYP2C9*4), rs28371685 (CYP2C9*11), rs72558187 (CYP2C9*13), rs35742686 (CYP2D6*3), rs5030656 (CYP2D6*9), rs1065852 (CYP2D6*10), rs16947 (CYP2D6*2), rs28371706 (CYP2D6*17), rs28371725 (CYP2D6*41), rs3892097 (CYP2D6*4), rs5030655 (CYP2D6*6), rs5030865 (CYP2D6*8 AND*14), rs59421388 (CYP2D6*29), rs774671100 (CYP2D6*15), rs5030862 (CYP2D6*12), rs5030863 (alternatively rs201377835, CYP2D6*11), rs5030867 (CYP2D6*7), CYP2D6 gene deletion, CYP2D6 gene multiplication, rs35599367 (CYP3A4*22), rs776746 (CYP3A5*3), rs10264272 (CYP3A5*6), rs41303343 (CYP3A5*7), DRD2 rs1799732, GRIK1 rs2832407, HLA-B*15:02 rs151107659, HLA-A*31:01, MC4R rs489693, MTHFR rs1801131 and rs1801133, OPRM1 rs1799971, SLC6A4 rs25531 and rs63749047, UGT1A4 rs2011425, and UGT2B15 rs1902023.

16. The method of claim 1, wherein the set of medications comprises a SSRI or a tricyclic antidepressant (TCA), a serotonin-norepinephrine reuptake inhibitor (SNRI), a sodium channel modulating agent, or an antipsychotic, wherein the SSRI is citalopram, fluvoxamine, paroxetine, escitalopram or sertraline, the TCA is amitriptyline, the SNRI is milnacipran or venlafzine, the sodium channel modulating agent is lamotrigine, and the antipsychotic is clozapine.

17. The method of claim 1, wherein the at least one neuropsychiatric disorder is depression, psychosis, or substance abuse.

18. The method of claim 1, wherein the first neuropsychiatric disorder is substance abuse and the first medication is methadone or bupropion, or wherein the first neuropsychiatric disorder is depression and the first medication is mertazapine.

19. A non-transitory computer readable storage medium and one or more computer programs embedded therein, the one or more computer programs comprising instructions for generating a report presenting subject-specific information relevant to a treatment of a neuropsychiatric disorder or associated comorbidity, the instructions which, when executed by a computer system, cause the computer system to:
obtain a set of genetic test results specific to a subject diagnosed with the neuropsychiatric disorder, wherein the set of genetic test results comprises allelic information for each gene in a set of genomic loci, wherein each respective genomic locus in the set of genomic loci is associated with at least one neuropsychiatric disorder in a plurality of neuropsychiatric disorders and associated comorbidities;
obtain a set of medications including a first medication for treatment of a first neuropsychiatric disorder in the plurality of neuropsychiatric disorders;
obtain a first patient-specific evaluation associated with the first medication using all or a portion of the set of genetic test results;
determine a set of alternative medications to the first medication for the treatment of the first neuropsychiatric disorder, wherein the set of alternative medications and the first medication belong to a common therapeutic class;
determine, for each respective alternative medication of the set of alternative medications, a corresponding alternative patient-specific evaluation using at least all or a portion of the set of genetic test results; and
generate the report, wherein the report comprises the first medication, the first patient-specific evaluation, the set of alternative medications and, for each alternative medication in the set of alternative medications, the corresponding alternative patient-specific evaluation.

20. A device for generating a report presenting subject-specific information relevant to a treatment of a neuropsychiatric disorder or associated comorbidity, the device comprising one or more processors, and memory storing one or more programs for execution by the one or more processors, the one or more programs comprising instructions for:
obtaining a set of medications including a first medication for treatment of a first neuropsychiatric disorder in the plurality of neuropsychiatric disorders;
obtaining a first patient-specific evaluation associated with the first medication using all or a portion of the set of genetic test results;
determining a set of alternative medications to the first medication for the treatment of the first neuropsychiatric disorder, wherein the set of alternative medications and the first medication belong to a common therapeutic class;
determining, for each respective alternative medication of the set of alternative medications, a corresponding alternative patient-specific evaluation using at least all or a portion of the set of genetic test results; and
generating the report, wherein the report comprises the first medication, the first patient-specific evaluation, the set of alternative medications and, for each alternative medication in the set of alternative medications, the corresponding alternative patient-specific evaluation.
